(19) United States
(12) Patent Application Publication
(10) Pub. No.: US 2025/0002959 A1
(43) Pub. Date: Jan. 2, 2025

(54) One-Step Method for Synthesis of Circular RNA

(71) Applicant: Suzhou Abogen Biosciences Co., Ltd., Suzhou, Jiangsu (CN)

(72) Inventors: Shaojun Qi, Suzhou, Jiangsu (CN); Huayuan Yan, Suzhou, Jiangsu (CN); Peng Gao, Suzhou, Jiangsu (CN)

(21) Appl. No.: 18/697,846

(22) PCT Filed: Sep. 12, 2023
(86) PCT No.: PCT/CN2023/118128

§ 371 Date: Apr. 2, 2024

(30) Foreign Application Priority Data
Sep. 13, 2022 (WO) PCT/CN2022/118337

(51) Int. Cl. C12P 19/34

(52) U.S. Cl. CPC C12P 19/34

(57) ABSTRACT

Provided are methods of preparing a circular RNA, comprising providing a template DNA, wherein the template DNA comprises a sequence encoding a precursor RNA, in a reaction solution to allow synthesis of precursor RNA by in vitro transcription of the template DNA and allowing the precursor RNA to self-splice, thereby producing a circular RNA, wherein the in vitro transcription of the template DNA and the self-splicing (i.e., circularization) of the precursor RNA are carried out in the same reaction solution under the same reaction conditions (e.g., the same reaction temperature). The method can be carried out in a single reaction vessel and does not require a step of purifying the precursor RNA before allowing the precursor RNA to self-splice.

### FIELD

The disclosure relates to circular RNA and to methods of making and using circular RNA.

### BACKGROUND

Messenger RNA (mRNA) is a type of single-stranded RNA involved in protein synthesis. In vitro transcribed (IVT) mRNAs have attracted much attention as novel agents with great therapeutic potential recently. Especially, the successful use of mRNA vaccines for COVID-19 has proven its safety and efficacy in vivo. However, the use of mRNA in nonvaccine therapies such as protein replacement is limited by several factors including mRNA stability, persistence of its expression in vivo, immunogenicity, and expressing cell types.

Circular RNA (circRNA) has emerged as a therapeutic agent. CircRNA is a type of single-stranded RNA which forms a 3′-5′ covalently closed loop. CircRNAs are created by a non-canonical splicing process termed “backsplicing”, whereby the spliceosome fuses a splice donor site in a downstream exon to a splice acceptor site in an upstream exon. Although circRNAs are generally noncoding, several studies have provided evidence that some circRNAs can be translated into proteins. Unlike linear mRNAs, circRNAs do not require a 5′-cap or 3′-poly (A) tail for its stability. Moreover, circRNAs have beneficial features not shared by mRNAs, such as reduced immunogenicity and extended translation duration. For these reasons, circRNAs have been explored as therapeutic agents.

Researchers have developed several methods to ligate the ends of linear RNA precursor into closed circular RNAs in vitro. The most commonly used methods include enzyme ligation and ribozyme ligation. Enzyme ligation-mediated circularization usually requires a complementary splint (a DNA or RNA oligo) to bring both ends of the RNA molecules closer and the ligation is catalyzed by enzymes from bacteriophage T4, such as T4 DNA ligase, T4 RNA ligase 1, and T4 RNA ligase 2. However, all these ligase-mediated circularizations are not efficient enough, especially for large RNA molecules. In addition, the generation of intermolecular end-joining by-products in the ligation reaction cannot be avoided entirely, leading to complicated system optimization and unfavorable production-scale-up.

Alternatively, circular RNAs can be made by ribozyme ligation. Ribozyme-mediated RNA circularization is commonly performed by the permuted intron and exon (PIE) method based on a modified group I intron self-splicing system. Group I introns are large self-splicing ribozymes. Native group I introns do not require assistance from the spliceosome or other proteins to self-splice but rely on magnesium and free guanosine nucleotides to initiate and complete the reaction. This process leads to the ligation of exons flanking introns and internal intron circularization to generate intronic circRNAs. Previous studies designed the PIE system using a modified group I intron, including placement of the 5′ half of the group I intron to the tail of the exon and transferring the remaining 3′ half to the head of the same exon (Puttaraju et al., 1992, Group I permuted intron-exon (PIE) sequences self-splice to produce circular exons”, Nucleic Acids Res 20, 5357-5364; Wesselhoeft et al., 2018, “Engineering circular RNA for potent and stable translation in eukaryotic cells, Nature Communications 9, 2629). This method achieves RNA circularization by a regular group I intron self-splicing reaction that includes two transesterifications at defined splice sites. Attack of the 5′ splice site by free GTP leads to the release of the 3′ end sequence (5′ half intron) of the PIE construct (first transesterification). The free 3′ OH group of the newly generated 3′ half exon attacks the 3′ splice site in the second transesterification reaction. This leads to the release of circRNA and 3′ half intron.

Compared with enzyme ligation-mediated circularization, the PIE method can be used to circularize larger linear RNA precursors, does not require additional protein ligase addition, and its reaction conditions and purification methods are relatively easier to develop and optimize. Circular RNAs encoding foreign proteins synthesized by the PIE method have been validated both in vitro and in vivo and retain the characteristics of low immunogenicity and longer translation duration, which broaden their applications (Wesselhoeft et al., 2019, “RNA circularization diminishes immunogenicity and can extend translation duration in vivo.” Mol. Cell. 74, 508-520; Qu et al., 2022, “Circular RNA vaccines against SARS-CoV-2 and emerging variants”, Cell 185, 1728-1744). Based on these advantages, the PIE system is currently the most studied and widely used method for RNA circularization.

The generation of circRNA in vitro by the PIE method requires a construct having a sequence of interest flanked by the 3′ and 5′ introns of the permuted group I catalytic intron. The PIE sequence vector is used as a template for in vitro transcription after being linearized by single enzyme digestion. Precursor RNAs are obtained by a T7 polymerase mediated standard in vitro transcription (IVT) reaction followed by purification. The yield of IVT reaction and the purity of purified products need to be optimized and guaranteed in the production process, and they are also the technical core of nucleic acid drug production. Pre-denaturation of purified precursor RNA at about 70° C. followed by rapid cooling down renaturation is required before proceeding to subsequent reactions to ensure efficient circularization. Special pre-denaturation equipment may be required in industrial scale-up production to achieve adequate and uniform heating for the larger reaction system and ensure a rapid and accurate cooling process. The optimal temperature for the circularization should be set at around 55° C. and it needs to be performed in a specific buffer system (containing 2 mM GTP, 50 mm Tris HCl, 10 mM MgCl₂, 1 mm DTT, pH 7.5). In addition, precursor RNA concentration needs to be optimized for circularization. When the precursor RNA concentration is low, it can be sufficiently circularized, but increasing the reaction volume exponentially in subsequent production scale-up poses great challenges to both reaction temperature control and reaction vessel selection. Although increasing the concentration of precursor RNA can decrease the reaction volume and alleviate the pressure for scale-up production, this comes at the expense of the rate of circularization. According to the well-established process flow, at least two days are required in scale-up production, from IVT of the linearized plasmid to precursor purification, accompanied by the potential loss of RNAs. The purified precursor RNA requires special denaturation and reaction equipment compatible with 55° C. condition for circularization, which is rapidly terminated followed by column chromatography purification. These steps require at least two days. Thus, conventional processes take at least four days to obtain purified circRNAs. There remain technical challenges and difficulties in scaling up the production of circRNAs in conventional two-step processes.

There is a need for ribozyme-mediated circularization processes that are simpler, faster, and more efficient than conventional processes.

### BRIEF SUMMARY

In an aspect, the invention provides a method of preparing a circular RNA (Method 1.0), comprising providing a template DNA, wherein the template DNA comprises a sequence encoding a precursor RNA, in a reaction solution to permit synthesis of the precursor RNA by in vitro transcription of the template DNA and allowing the precursor RNA to self-splice, thereby producing a circular RNA, wherein the in vitro transcription of the template DNA and the self-splicing (i.e., circularization) of the precursor RNA are carried out in the same reaction solution under the same reaction conditions (e.g., the same reaction temperature). The method can be carried out in a single step in a single reaction vessel and does not require a step of purifying the precursor RNA before allowing the precursor RNA to self-splice.

In some embodiments, the DNA template comprises the following elements operably connected to each other and arranged in the following sequence: an RNA polymerase promoter, optionally a 5′ homology arm, a 3′ Group I intron fragment containing a 3′ splice site dinucleotide, optionally a 5′ spacer sequence, an insert sequence, optionally a 3′ spacer sequence, a 5′ Group I intron fragment containing a 5′ splice site dinucleotide, and optionally a 3′ homology arm. In some embodiments, the insert sequence comprises a protein coding sequence, optionally wherein the insert sequence comprises an IRES (internal ribosomal entry site) sequence operably connected to the protein coding sequence.

In some embodiments, the reaction solution comprises Mg²⁺ at the concentration greater than 26 mM, e.g., greater than 30 mM or greater than 35 mM. In certain embodiments, the concentration of Mg²⁺ in the solution is from 38 mM to 66 mM.

In some embodiments, the reaction solution comprises a pyrophosphatase at the concentration of from 1 U/ml to 5 U/ml, e.g., from 1 U/ml to 4 U/ml, from 1.5 U/ml to 3 U/ml, from 1.5 U/ml to 2.5 U/ml, about 1 U/ml, about 2 U/ml, or about 4 U/ml.

In some embodiments, the reaction solution comprises 38-66 mM Mg²⁺, optionally 1-4 U/ml pyrophosphatase, an RNA polymerase, a RNase inhibitor, ATP, GTP, CTP, UTP, DTT, and a monovalent cation (Na⁺ or K⁺).

In some embodiments, the in vitro transcription of the template DNA and the circularization (i.e., self-splicing) of the precursor RNA are carried out at a temperature of from 37° C. to 55° C. In some embodiments, the in vitro transcription of the template DNA and the circularization (i.e., self-splicing) of the precursor RNA are carried out at a temperature higher than 37° C., e.g., from 39° C. to 55° C., from 41° C. to 55° C., from 43° C. to 55° C., from 39° C. to 50° C., from 41° C. to 50° C., from 43° C. to 50° C., from 39° C. to 47° C., from 41° C. to 47° C., from 43° C. to 47° C., from 47° C. to 55° C., from 50° C. to 55° C., from 39° C. to 43° C., about 39° C., about 41° C., about 43° C., about 47° C., about 53° C., or about 55° C.

In some embodiments, the in vitro transcription of the template DNA and the circularization (i.e., self-splicing) of the precursor RNA are carried out for at least 1 hour, e.g., at least 1.5 hours, at least 2.5 hours, at least 3 hours, from 1 hour to 3 hours, from 1.5 hours to 3 hours, from 2 hours to 3 hours, or from 2.5 hours to 3 hours. In certain embodiments, the in vitro transcription of the template DNA and the circularization (i.e., self-splicing) of the precursor RNA are carried out for 2.5-3 hours.

In some embodiments, the method further comprises a step of removing the DNA template after the self-splicing of the RNA, optionally wherein the DNA template is removed by adding a DNase I, e.g., for 30 min at 37° C.

In some embodiments, the method the method further comprises a step of purifying the circular RNA after the self-splicing of the RNA or after the step of removing the DNA template if the method comprises a step of removing the DNA template. In some embodiments, the purification step is selected from a precipitation step, a tangential flow filtration step and a chromatographic step, and a combination thereof.

In another aspect, the invention provides a reaction solution for one-step circular RNA synthesis, comprising Mg²⁺ in a concentration of greater than 26 mM (e.g., from 38 mM to 66 mM), optionally 1-4 U/ml pyrophosphatase, a RNA polymerase, an RNase inhibitor, ATP, GTP, CTP, UTP, DTT, and a monovalent cation (Na⁺ or K⁺).

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF DRAWINGS

FIG. 1 shows the schematic diagram of the circRNA precursor used in the Examples.

FIG. 2 shows schematic diagrams of the conventional procedure (left) and one-step procedure (right).

FIG. 3 shows the fragment analysis for the products of circularization with or without pre-denaturation. Peaks corresponding to intronic sequences from both ends of the precursor, circularized RNAs (represented by a circle), and remained precursors (represented by a curve) are shown. The proportions of circular RNA and precursor are labeled.

FIG. 4A shows the effect of Mg²⁺ concentrations on circularization efficiency (% circRNA in total) in the IVT system. The dotted line indicates the 40% circularization rate.

FIG. 4B shows the fragment analysis for the products of IVT with different Mg²⁺ concentrations. Peaks corresponding to intronic sequences, circularized RNAs (represented by a circle), and remained precursors (represented by a curve) are shown.

FIG. 4C shows the effect of Mg²⁺ concentrations on the yield (total RNA) in the IVT system. The dotted line indicates 200 ug yield.

FIG. 5A shows the circularization efficiency at different reaction durations. The dotted line indicates the 40% circularization rate.

FIG. 5B shows the yield (total RNA) in the IVT system at different reaction durations. The dotted line indicates a 200 ug yield.

FIG. 6A shows the Pareto chart of the standardized effects of different factors or combinations of specific factors on yield (total RNA) in the IVT system. On the Pareto chart, bars that cross the reference line are statistically significant.

FIG. 6B shows the main effects plot for yield (total RNA)/reaction.

FIG. 7 shows the interaction plot showing the mean yield (total RNA) versus temperature for the two T7 RNAP types.

FIG. 8A shows the Pareto chart of the standardized effects of different factors or combinations of specific factors on circularization efficiency. On the Pareto chart, bars that cross the reference line are statistically significant.

FIG. 8B shows the main effects plot for circularization efficiency/reaction.

FIG. 9 shows the interaction plot showing the mean circularization efficiency versus Mg²⁺ concentration for the three pyrophosphatase concentrations.

FIG. 10 shows the line chart of % dsRNAs in the one-step IVT reaction products at different temperatures and Mg²⁺ concentrations.

### DETAILED DESCRIPTION

The following description of the preferred embodiment(s) is merely exemplary in nature and is in no way intended to limit the disclosure, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Conventional PIE-based circularization processes comprise two separate steps: in vitro transcription (IVT) step and circularization step. According to the previous study (Wesselhoeft et al., (2018), “Engineering circular RNA for potent and stable translation in eukaryotic cells, Nature Communications 9, 2629), the circularization of IVT-derived precursor RNAs through the self-splicing process of group I intron requires 2 mM GTP and 10 mM MgCl₂, while the conventional IVT reaction solution contains 10 mM GTP and 26 mM MgCl₂. It has been found that precursor RNAs are not circularized efficiently in the conventional IVT condition (10 mM GTP and 26 mM MgCl₂). Therefore, the conventional IVT condition need to be optimized for one-step reaction system for circular RNA synthesis. In the present invention, it has been found that increasing Mg²⁺ concentration from 26 mM to 36 mM not only promotes the circularization of the circRNA precursor but also increase the IVT yield. In the present invention, the conditions for the one step reaction system have been further optimized. For example, it has been found that the addition of pyrophosphatase to the reaction solution increases the IVT yield and also increases the circularization efficiency. It is believed that it is because pyrophosphatase helps maintain the magnesium ion concentration stable throughout the IVT/circularization process. After multifactorial optimization, the one-step process can achieve the circularization efficiency similar to the conventional process and reduce double-stranded RNAs (dsRNAs) production.

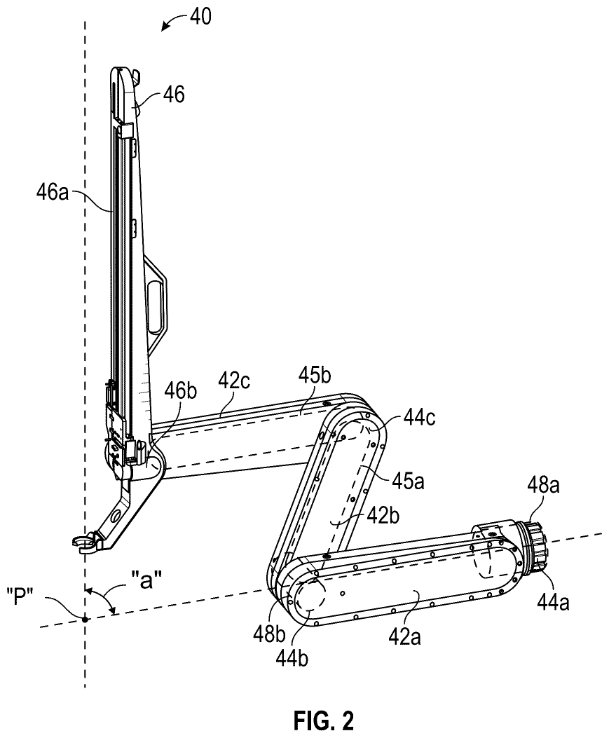

The one-step circular RNA synthesis system of the present invention has significant advantages over conventional processes for the synthesis of circular RNA, such as increased circularization efficiency, decreased by-products that may cause inflammation (e.g., dsRNA), and shortened production cycle, which is beneficial for process scale-up and technology transfer. The synthesis of circRNAs using the conventional process first requires IVT to generate the precursors and after purification, the circularization of the purified precursors requires the additional introduction of GTP and buffer containing Mg²⁺ to trigger circularization. Moreover, more than 70% circularization efficiency is achieved only when the precursor is pre-denatured at 70° C. before circularization. So conventional processes require five steps, including IVT, 1^{st} purification, pre-denaturation, circularization, and 2^{nd} purification in sequence to obtain the final circRNAs (FIG. 2). In contrast, IVT and circularization are carried out in one step in the method of the present invention. Moreover, the one-step process does not require a pre-denaturation step before circularization. Therefore, the one-step process, which does not require special denaturation equipment and a high-temperature reaction condition of 55 degrees, is significantly simpler than conventional processes and shortens the circRNA synthesis cycle from 4 days to 2 days (FIG. 2).

The invention provides, in an aspect, a method of preparing a circular RNA (Method 1.0), comprising providing a template DNA, wherein the template DNA comprises a sequence encoding a precursor RNA, in a reaction solution to permit synthesis of the precursor RNA by in vitro transcription of the template DNA and allowing the precursor RNA to self-splice, thereby producing a circular RNA, wherein the in vitro transcription of the template DNA and the self-splicing (i.e., circularization) of the precursor RNA are carried out in the same reaction solution under the same reaction conditions (e.g., the same reaction temperature).

For example, the invention includes:
1.1. Method 1.0, wherein the method does not comprise a step of purifying the precursor RNA before allowing the precursor RNA to self-splice.
1.2. Method 1.0 or 1.1, wherein the template DNA is circular, optionally wherein the circular template DNA is a DNA plasmid.
1.3. Method 1.0 or 1.1, wherein the template DNA is linear, optionally wherein the linear template DNA is prepared by linearizing a DNA plasmid, e.g., by a restriction enzyme.
1.4. Any of preceding methods, wherein the DNA template comprises the following elements operably connected to each other and arranged in the following sequence: an RNA polymerase promoter, optionally a 5′ homology arm, a 3′ Group I intron fragment containing a 3′ splice site dinucleotide, optionally a 5′ spacer sequence, an insert sequence, optionally a 3′ spacer sequence, a 5′ Group I intron fragment containing a 5′ splice site dinucleotide, and optionally a 3′ homology arm.
1.5. Method of 1.4, wherein the insert sequence comprises a noncoding sequence having a biological activity, optionally wherein the noncoding sequence is micro RNA or lnc (long noncoding) RNA.
1.6. Method of 1.4, wherein the insert sequence comprises a protein coding sequence, optionally wherein the insert sequence comprises an IRES sequence operably connected to the protein-coding sequence.
1.7. Method of 1.6, wherein the protein-coding sequence encodes an antibody.
1.8. Method of 1.6-1.7, wherein the IRES sequence is selected from an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2, Human rhinovirus B, Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Human enterovirus B, Equine rhinitis virus, Ectropis obliqua picoma-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-l, Human BCL2, Human BiP, Human c-IAPl, Human c-myc, Human eIF4G, Mouse NDST4L, Human LEF1, Mouse HIF1 alpha, Human n-myc, Mouse Gtx, Human p27kipl, Human PDGF2/c-sis, Human p53, Human Pim-1, Mouse Rbm3, Drosophila reaper, Canine Scamper, Drosophila Ubx, Salivirus, Cosavirus, Parechovirus, Human UNR, Mouse UtrA, Human VEGF-A, Human XIAP, Drosophila hairless, S. cerevisiae TFIID, S. cerevisiae YAP1, Human c-src, Human FGF-1, Simian picomavirus, Turnip crinkle virus, an aptamer to eIF4G, Coxsackievirus B3 (CVB3) or Coxsackievirus A (CVB1/2).
1.9. Method of 1.8, wherein the IRES sequence is an IRES sequence of Coxsackievirus B3 (CVB3).
1.10. Any of preceding methods, wherein the RNA polymerase promoter is a T7 virus RNA polymerase promoter, T6 virus RNA polymerase promoter, SP6 virus RNA polymerase promoter, T3 virus RNA polymerase promoter, or T4 virus RNA polymerase promoter.
1.11. Any of preceding methods, wherein the RNA polymerase promoter is a T7 virus RNA polymerase promoter.
1.12. Any of preceding methods, wherein the 3′ group I intron fragment is a contiguous sequence that is at least 80% (e.g., at least 85%, at least 90%, at least 95%, or 100%) homologous to a 3′ proximal fragment of a natural group I intron
1.13. Any of preceding methods, wherein the 5′ group I intron fragment is a contiguous sequence that is at least 80% (e.g., at least 85%, at least 90%, at least 95%, or 100%) homologous to a 5′ proximal fragment of a natural group I intron.
1.14. Methods 1.12-1.13, wherein the natural group I intron is derived from T4 bacteriophage gene td or Cyanobacterium Anabaena sp. pre-tRNA-Leu gene.
1.15. Any of preceding methods, wherein the 5′ homology arm and 3′ homology arm are perfectly complement to each other.
1.16. Any of Methods 1.0-1.14, wherein the 5′ homology arm and 3′ homology arm are partially (e.g., at least 80%, at least 85%, at least 90%, or at least 95%) complement to each other.
1.17. Any of preceding methods, wherein the 5′ homology arm is from 5 to 50 nucleotides in length, e.g., from 9 to 19 nucleotides in length.
1.18. Any of preceding methods, wherein the 3′ homology arm is from 5 to 50 nucleotides in length, e.g., from 9 to 19 nucleotides in length.
1.19. Any of preceding methods, wherein the DNA template comprises a 5′ spacer sequence between the 3′ Group I intron fragment and the insert sequence, optionally wherein the 5′ spacer sequence is from 5 to 50 nucleotides in length, e.g., from 10 to 20 nucleotides in length.
1.20. Any of preceding methods, wherein the 5′ spacer sequence is a polyA sequence or a polyA-C sequence.
1.21. Any of preceding methods, wherein the DNA template comprises a 3′ spacer sequence between the insert sequence and the 5′ Group I intron fragment, optionally wherein the 3′ spacer sequence is from 5 to 50 nucleotides in length, e.g., from 10 to 20 nucleotides in length.
1.22. Any of preceding methods, wherein the 3′ spacer sequence is a polyA sequence or a polyA-C sequence.
1.23. Any of preceding methods, wherein the reaction solution comprises Mg²⁺ at the concentration greater than 26 mM, e.g., greater than 30 mM or greater than 35 mM.
1.24. Any of preceding methods, wherein the concentration of Mg²⁺ in the solution is from 30 mM to 100 mM, e.g., from 30 mM to 90 mM, from 30 mM to 80 mM, from 30 mM to 70 mM, from 30 mM to 60 mM, from 30 mM to 50 mM, from 30 mM to 40 mM, from 35 mM to 100 mM, from 35 mM to 90 mM, from 35 mM to 80 mM, from 35 mM to 70 mM, from 35 mM to 60 mM, from 35 mM to 50 mM, from 35 mM to 40 mM, from 38 to 66 mM, e.g., about 38 mM, optionally wherein the concentration of Mg²⁺ in the solution is from 38 mM to 66 mM.
1.25. Any of preceding methods, wherein the reaction solution comprises a pyrophosphatase at the concentration of from 1 U/ml to 5 U/ml, e.g., from 1 U/ml to 4 U/ml, from 1.5 U/ml to 3 U/ml, from 1.5 U/ml to 2.5 U/ml, about 1 U/ml, about 2 U/ml, or about 4 U/ml.
1.26. Any of preceding methods, wherein the reaction solution comprises an RNA polymerase, an RNase inhibitor, ATP, GTP, CTP, UTP, DTT, and a monovalent cation (Na⁺ or K⁺).
1.27. Any of preceding methods, wherein the reaction solution comprises 5 U/μl RNA polymerase, 1 U/μl RNase inhibitor, 10 mM ATP, 10 mM GTP, 10 mM CTP, 10 mM UTP, 10 mM DTT, and 5 mM monovalent cation (Na⁺ or K⁺).
1.28. Any of preceding methods, wherein the reaction solution comprises 38-66 mM Mg²⁺ optionally 1-4 U/ml pyrophosphatase, an RNA polymerase, an RNase inhibitor, ATP, GTP, CTP, UTP, DTT, and a monovalent cation (Na⁺ or K⁺).
1.29. Any of preceding methods, wherein the reaction solution comprises 38 mM Mg²⁺, 2 U/ml pyrophosphatase, an RNA polymerase, an RNase inhibitor, ATP, GTP, CTP, UTP, DTT, and a monovalent cation (Na⁺ or K⁺).
1.30. Any of preceding methods, wherein the reaction solution comprises 38-66 mM Mg²⁺, optionally 1-4 U/ml pyrophosphatase, 5 U/μl RNA polymerase, 1 U/μl RNase inhibitor, 10 mM ATP, 10 mM GTP, 10 mM CTP, 10 mM UTP, 10 mM DTT, and 5 mM monovalent cation (Na⁺ or K⁺).
1.31. Any of preceding methods, wherein the reaction solution comprises 38 mM Mg²⁺, 2 U/ml pyrophosphatase, 5 U/μl RNA polymerase, 1 U/μl RNase inhibitor, 10 mM ATP, 10 mM GTP, 10 mM CTP, 10 mM UTP, 10 mM DTT, and 5 mM monovalent cation (Na⁺ or K⁺).
1.32. Any of preceding methods, wherein the reaction solution comprises a buffer.
1.33. Any of preceding methods, wherein the pH of the reaction solution is from 6 to 8, e.g., from 7 to 8, or about 7.5.
1.34. Any of preceding methods, wherein the reaction solution comprises a RNA polymerase selected from T7 virus RNA polymerase, T6 virus RNA polymerase, SP6 virus RNA polymerase, T3 virus RNA polymerase, or T4 virus RNA polymerase.
1.35. Any of preceding methods, wherein the RNA polymerase promoter in the DNA template is a T7 virus RNA polymerase promoter and the reaction solution comprises a T7 virus RNA polymerase.
1.36. Any of preceding methods, wherein the in vitro transcription of the template DNA and the circularization (i.e., self-splicing) of the precursor RNA are carried out at a temperature of from 37° C. to 55° C., e.g., from 39° C. to 55° C., from 41° C. to 55° C., from 43° C. to 55° C., from 37° C. to 50° C., from 39° C. to 50° C., from 41° C. to 50° C., from 43° C. to 50° C., from 37° C. to 47° C., from 39° C. to 47° C., from 41° C. to 47° C., from 43° C. to 47° C., from 47° C. to 55° C., from 50° C. to 55° C., from 39° C. to 43° C., about 37° C., about 39° C., about 41° C., about 43° C., about 47° C., about 53° C., or about 55° C., optionally wherein the in vitro transcription of the template DNA and the circularization (i.e., self-splicing) of the precursor RNA are carried out at a temperature higher than 37° C., e.g., from 39° C. to 55° C., from 41° C. to 55° C., from 43° C. to 55° C., from 39° C. to 50° C., from 41° C. to 50° C., from 43° C. to 50° C., from 39° C. to 47° C., from 41° C. to 47° C., from 43° C. to 47° C., from 47° C. to 55° C., from 50° C. to 55° C., from 39° C. to 43° C., about 39° C., about 41° C., about 43° C., about 47° C., about 53° C., or about 55° C.
1.37. Any of preceding methods, wherein the in vitro transcription of the template DNA and the circularization (i.e., self-splicing) of the precursor RNA are carried out at a temperature of from 47° C. to 55° C., e.g., from 50° C. to 55° C., about 47° C., about 53° C., or about 55° C. and the RNA polymerase is a thermostable polymerase (e.g., T7 Toyobo).
1.38. Any of preceding methods, wherein the in vitro transcription of the template DNA and the circularization (i.e., self-splicing) of the precursor RNA are carried out for at least 1 hour, e.g., at least 1.5 hours, at least 2.5 hours, at least 3 hours, from 1 hour to 3 hours, from 1.5 hours to 3 hours, from 2 hours to 3 hours, or from 2.5 hours to 3 hours, optionally wherein the in vitro transcription of the template DNA and the circularization (i.e., self-splicing) of the precursor RNA are carried out for 2.5-3 hours.
1.39. Any of preceding methods, wherein the method further comprises a step of removing the DNA template after synthesis of the precursor RNA, optionally wherein the DNA template is removed by adding a DNase I, e.g., for 30 min at 37° C.
1.40. Any of preceding methods, wherein the method further comprises a step of purifying the circular RNA thus synthesized, e.g., after the step of removing the DNA template if the method comprises a step of removing the DNA template.
1.41. Method of 1.40, wherein the purification step is selected from a precipitation step, a tangential flow filtration step and a chromatographic step, and a combination thereof.
1.42. Method of 1.41, wherein the precipitation step is an alcoholic precipitation step or LiCl precipitation, optionally wherein the precipitation step is LiCl precipitation.
1.43. Method of 1.41, wherein the tangential flow filtration step is a diafiltration step using tangential flow filtration and/or a concentration step using tangential flow filtration.
1.44. Method of 1.41, wherein the chromatographic step is selected from HPLC, anion exchange chromatography, affinity chromatography, hydroxyapatite chromatography, magnetic bead chromatography, and core bead chromatography, optionally wherein the chromatographic step is magnetic bead chromatography.
1.45. Any of preceding methods, wherein the precursor RNA is unmodified, i.e., contains only naturally-occurring nucleosides, e.g., contains adenosine, guanosine, cytidine and uridine.
1.46. Any of Methods 1.0-1.44, wherein the precursor RNA is partially modified or completely modified, i.e., contains nucleosides other than or in addition to adenosine, guanosine, cytidine and uridine.
1.47. Method 1.46 wherein the precursor RNA comprises nucleosides selected from pseudouridine, 1-methylpseudouridine, 2-thiouridine, 4-thiouridine, 5-methylcytidine, N6-methyladenosine, and a combination thereof
1.48. Method 1.46, wherein a part or all of the ribonucleoside triphosphates in the reaction solution comprise ribonucleoside triphosphates other than or in addition to adenosine triphosphate (ATP), guanosine triphosphate (GTP), cytidine triphosphate (CTP) and uridine triphosphate (UTP).
1.49. Method 1.46 wherein a part or all of the ribonucleoside triphosphates in the reaction solution comprise modified nucleoside triphosphates, e.g., wherein the modified nucleoside triphosphates are selected from pseudouridine-5′-triphosphate, 1-methylpseudouridine-5′-triphosphate, 2-thiouridine-5′-triphosphate, 4-thiouridine-5′-triphosphate, 5-methylcytidine-5′-triphosphate, N6-methyladenosine-5′-triphosphate and a combination thereof.
1.50. Method 1.46 wherein the nucleosides in the precursor RNA do not comprise uridine, but comprise nucleosides selected from pseudouridine, 1-methylpseudouridine, 2-thiouridine, 4-thiouridine, and a combination thereof.
1.51. Method 1.46 wherein the nucleosides in the precursor RNA do not comprise cytidine, but comprise 5-methylcytidine.
1.52. Any foregoing Method wherein the circularization efficiency is at least 70%
1.53. Any foregoing Method wherein the percentage of dsRNA relative to total RNA in the final product is less than 1%, e.g., less than 0.10%.
1.54. Any foregoing Method wherein the reaction solution is a reaction solution in accordance with Reaction Solution 2, et seq., as set forth below.

In an embodiment, the reaction solution of the method according to the present disclosure comprises Mg²⁺. The reaction solution may further comprise a pyrophosphatase.

In a further embodiment, the reaction solution may comprise nucleoside triphosphates.

In yet a further embodiment, the reaction solution may comprise a reducing agent.

In yet another embodiment, the reaction solution may comprise an RNA polymerase.

In another embodiment, the reaction solution may comprise an RNase inhibitor.

In another embodiment, the reaction may comprise a monovalent cation.

The selections and/or concentrations of each of the components are as described herein. The concentrations are based on the reaction solution/mixture as used in the method.

For example, the concentration of Mg²⁺ in the solution is greater than 26 mM, particularly is from 38 to 66 mM, more particularly is 38 mM. For example, the concentration of pyrophosphatase in the solution is from 1 U/ml to 5 U/ml, particularly 1 U/ml to 4 U/ml, more particularly 2 U/ml. For example, the nucleoside triphosphates comprise adenosine triphosphate (ATP), guanosine triphosphate (GTP), cytidine triphosphate (CTP) and uridine triphosphate (UTP) and the concentration of each of them is 10 mM. For example, the reducing agent is DTT and its concentration is 10 mM. For example, the RNA polymerase is a T7 virus polymerase and its concentration is 5 U/μl. For example, the concentration of RNase inhibitor is 1 U/μl. For example, the monovalent cation is Na⁺ or K⁺ and its concentration is 5 mM.

In an embodiment, the reaction solution of the method according to the present disclosure comprises Mg²⁺, an RNA polymerase, an RNase inhibitor, nucleoside triphosphates, a reducing agent, and a monovalent cation (Na+ or K+) with optionally a pyrophosphatase.

In an embodiment, the reaction conditions of the method according to the present disclosure comprise the temperature of in vitro transcription of the template DNA and the circularization (i.e., self-splicing) of the precursor RNA.

In a further embodiment, the reaction conditions of the method according to the present disclosure comprise the duration of in vitro transcription of the template DNA and the circularization (i.e., self-splicing) of the precursor RNA.

The selection of each of the conditions are as described herein. For example, the in vitro transcription of the template DNA and the circularization (i.e., self-splicing) of the precursor RNA are carried out at a temperature of from 37° C. to 55° C., particularly 39° C. to 50° C. or 47° C. to 55° C. with a thermostable RNA polymerase (e.g., T7 Toyobo). For example, the in vitro transcription of the template DNA and the circularization (i.e., self-splicing) of the precursor RNA are carried out for 2.5-3 hours.

In an embodiment, the reaction conditions of the method according to the present disclosure comprise the temperature and duration of in vitro transcription of the template DNA and the circularization (i.e., self-splicing) of the precursor RNA.

In the most particular embodiment, the reaction solution of the method according to the present disclosure comprises 38 mM Mg²+, 2 U/ml pyrophosphatase, 5 U/μl RNA polymerase, 1 U/μl RNase inhibitor, 10 mM ATP, 10 mM GTP, 10 mM CTP, 10 mM UTP, 10 mM DTT, and 5 mM monovalent cation (Na⁺ or K⁺); the in vitro transcription of the template DNA and the circularization (i.e., self-splicing) of the precursor RNA are carried out at a temperature of from 37° C. to 55° C. and are carried out for 2.5-3 hours.

The disclosure further provides a circular RNA obtained by any of Methods 1, et seq.

The disclosure further provides a pharmaceutical composition comprising a circular RNA obtained by any of Methods 1, et seq.

The disclosure further provides a reaction solution for one-step circular RNA synthesis, which is an aqueous solution comprising Mg²⁺ in a concentration of greater than 26 mM, a pyrophosphatase, an RNA polymerase, an RNase inhibitor, nucleoside triphosphates, a reducing agent, and a monovalent cation (e.g., selected from Na⁺, K⁺ and combinations thereof)(Reaction Solution 2), e.g.,
2.1. Reaction Solution 2, wherein the reaction solution comprises Mg²⁺ at the concentration greater than 30 mM or greater than 35 mM.
2.2. Any preceding reaction solutions, wherein the concentration of Mg²⁺ in the solution is from 30 mM to 100 mM, e.g., from 30 mM to 90 mM, from 30 mM to 80 mM, from 30 mM to 70 mM, from 30 mM to 60 mM, from 30 mM to 50 mM, from 30 mM to 40 mM, from 35 mM to 100 mM, from 35 mM to 90 mM, from 35 mM to 80 mM, from 35 mM to 70 mM, from 35 mM to 60 mM, from 35 mM to 50 mM, from 35 mM to 40 mM, from 38 to 66 mM, e.g., about 38 mM, optionally wherein the concentration of Mg²⁺ in the solution is from 38 mM to 66 mM.
2.3. Any preceding reaction solutions, wherein the reaction solution comprises a pyrophosphatase at the concentration of from 1 U/ml to 5 U/ml, e.g., from 1 U/ml to 4 U/ml, from 1.5 U/ml to 3 U/ml, from 1.5 U/ml to 2.5 U/ml, about 1 U/ml, about 2 U/ml, or about 4 U/ml.
2.4. Any preceding reaction solutions wherein the nucleoside triphosphates are selected from the group consisting of ATP, GTP, CTP, UTP, pseudouridine-5′-triphosphate, 1-methylpseudouridine-5′-triphosphate, 2-thiouridine-5′-triphosphate, 4-thiouridine-5′-triphosphate, 5-methylcytidine-5′-triphosphate, N6-methyladenosine-5′-triphosphate, and combinations thereof.
2.5. Any preceding reaction solutions, wherein the nucleoside triphosphates comprise ATP, GTP, CTP, and UTP.
2.6. Any preceding reaction solutions, wherein the reaction solution comprises a pyrophosphatase at the concentration of from 1 U/ml to 5 U/ml, e.g., from 1 U/ml to 4 U/ml, from 1.5 U/ml to 3 U/ml, from 1.5 U/ml to 2.5 U/ml, about 1 U/ml, about 2 U/ml, or about 4 U/ml.
2.7. Any preceding reaction solutions, wherein the nucleoside triphosphates comprise pseudouridine-5′-triphosphate, ATP, GTP, and CTP.
2.8. Any preceding reaction solutions, wherein the nucleoside triphosphates comprise 1-methylpseudouridine-5′-triphosphate, ATP, GTP, and CTP.
2.9. Any preceding reaction solution wherein the reducing agent is DTT.
2.10. Any preceding reaction solutions, wherein the nucleoside triphosphates comprise pseudouridine-5′-triphosphate, 1-methylpseudouridine-5′-triphosphate, 2-thiouridine-5′-triphosphate, 4-thiouridine-5′-triphosphate, 5-methylcytidine-5′-triphosphate, N6-methyladenosine-5′-triphosphate or combinations thereof.
2.11. Any preceding reaction solutions, wherein the reaction solution comprises 5 U/μl RNA polymerase, 1 U/μl RNase inhibitor, 10 mM ATP, 10 mM GTP, 10 mM CTP, 10 mM UTP, 10 mM DTT, and 5 mM monovalent cation (Na⁺ or K⁺).
2.12. Any preceding reaction solutions, wherein the reaction solution comprises 38-66 mM Mg²⁺, optionally 1-4 U/ml pyrophosphatase, an RNA polymerase, an RNase inhibitor, ATP, GTP, CTP, UTP, DTT, and a monovalent cation (Na⁺ or K⁺).
2.13. Any preceding reaction solutions, wherein the reaction solution comprises 38 mM Mg²⁺, 2 U/ml pyrophosphatase, an RNA polymerase, an RNase inhibitor, ATP, GTP, CTP, UTP, DTT, and a monovalent cation (Na⁺ or K⁺).
2.14. Any preceding reaction solutions, wherein the reaction solution comprises 38-66 mM Mg²⁺, optionally 1-4 U/ml pyrophosphatase, 5 U/μl RNA polymerase, 1 U/μl RNase inhibitor, 10 mM ATP, 10 mM GTP, 10 mM CTP, 10 mM UTP, 10 mM DTT, and 5 mM monovalent cation (Na⁺ or K⁺).
2.15. Any preceding reaction solutions, wherein the reaction solution comprises 38 mM Mg²⁺, 2 U/ml pyrophosphatase, 5 U/μl RNA polymerase, 1 U/μl RNase inhibitor, 10 mM ATP, 10 mM GTP, 10 mM CTP, 10 mM UTP, 10 mM DTT, and 5 mM monovalent cation (Na⁺ or K⁺).
2.16. Any preceding reaction solutions, wherein the reaction solution comprises a buffer.
2.17. Any of preceding reaction solutions, wherein the pH of the reaction solution is from 6 to 8, e.g., from 7 to 8, or about 7.5.
2.18. Any preceding reaction solutions, wherein the reaction solution comprises an RNA polymerase selected from T7 virus RNA polymerase, T6 virus RNA polymerase, SP6 virus RNA polymerase, T3 virus RNA polymerase, or T4 virus RNA polymerase.

The disclosure further provides the use of any of Reaction Solutions 2, et seq. in a method of one-step circular RNA synthesis, e.g., in a method according to any of Methods 1, et seq.

In the method of the present invention, a precursor RNA is synthesized by in vitro transcription of a template DNA. The DNA template comprises a promoter upstream of the region that encodes the precursor RNA. The promoter is recognized by an RNA polymerase, for example a T7 promoter, which is recognized by T7 virus RNA polymerase. In some embodiments, the promoter is a T7 promoter and the RNA polymerase is a T7 virus RNA polymerase; or the promoter is a T6 promoter, and the polymerase is a T6 virus RNA polymerase; or the promoter is an SP6 virus RNA polymerase promoter and the polymerase is SP6 virus RNA polymerase; or the promoter is T3 virus RNA polymerase promoter and the polymerase is T3 virus RNA polymerase; or the promoter is T4 virus RNA polymerase promoter and the polymerase is T4 virus RNA polymerase. In certain embodiments, the RNA polymerase promoter is a T7 virus RNA polymerase promoter and the polymerase is a T7 virus RNA polymerase.

The template DNA may be linear or circular. In some embodiments, the template DNA is prepared by linearizing a DNA plasmid, e.g., by a restriction enzyme. In other embodiments, the template is circular (e.g., a DNA plasmid). The template DNA may comprise an RNA polymerase terminator sequence element downstream of the region that encodes the precursor RNA, especially when the template DNA is circular.

The template DNA comprises a sequence encoding a precursor RNA. As used herein, “circular precursor RNA” or “precursor RNA” refers to a linear RNA molecule that can self-splice, thereby producing a circular RNA (circRNA). The precursor RNA contains the circRNA sequence plus splicing sequences (e.g., intron fragments and optional 5′ and 3′ homology arms) necessary to circularize the RNA. These splicing sequences are removed from the precursor RNA during the circularization. The precursor RNA may be unmodified, partially modified or completely modified. In some embodiments, the precursor RNA is unmodified, i.e., the nucleoside moieties in the precursor RNA are naturally-occurring nucleosides, e.g., adenosine, guanosine, cytidine and uridine. In other embodiments, the precursor RNA is modified, i.e., the nucleoside moieties in the precursor RNA comprise nucleosides in addition to or in place of adenosine, guanosine, cytidine and uridine; for example the nucleosides comprise pseudouridine, 1-methylpseudouridine, 2-thiouridine, 4-thiouridine, 5-methylcytidine, N6-methyladenosine, or a combination thereof, for example where uridine is replaced with pseudouridine, 1-methylpseudouridine, 2-thiouridine, 4-thiouridine, and/or cytidine is replaced with 5-methylcytidine, and/or adenosine is replaced with N6-methyladenosine.

In some embodiments, the DNA template comprises the following elements operably connected to each other and arranged in the following sequence: a promoter recognized by an RNA polymerase, optionally a 5′ homology arm, a 3′ Group I intron fragment containing a 3′ splice site dinucleotide, optionally a 5′ spacer sequence, an insert sequence which comprises a sequence of interest, optionally a 3′ spacer sequence, a 5′ Group I intron fragment containing a 5′ splice site dinucleotide, and optionally a 3′ homology arm. As used herein, the phrase “operably connected” means that the elements are positioned on the DNA template such that a precursor RNA can be synthesized by in vitro transcription of the template DNA and the precursor RNA can then be circularized into a circular RNA using the methods disclosed herein.

In some embodiments, the 3′ group I intron fragment is a contiguous sequence that is at least 80% (e.g., at least 85%, at least 90%, at least 95%, or 100%) homologous to a 3′ proximal fragment of a natural group I intron, including the 3′ splice site dinucleotide, and optionally the adjacent exon sequence of at least 1 nucleotide in length (e.g., at least 5 nucleotides in length, at least 10 nucleotides in length, at least 15 nucleotides in length, at least 20 nucleotides in length, at least 25 nucleotides in length, or at least 50 nucleotides in length).

In some embodiments, the 5′ group I intron fragment is a contiguous sequence that is at least 80% (e.g., at least 85%, at least 90%, at least 95%, or 100%) homologous to a 5′ proximal fragment of a natural group I intron, including the 5′ splice site dinucleotide and, optionally, the adjacent exon sequence of at least 1 nucleotide in length (e.g., at least 5 nucleotides in length, at least 10 nucleotides in length, at least 15 nucleotides in length, at least 20 nucleotides in length, at least 25 nucleotides in length, at least 50 nucleotides in length).

The natural group I intron may be chosen from any group I intron that is known to self-splice. Examples of Group I intron include, but are not limited to, group I introns derived from T4 bacteriophage gene td or Cyanobacterium anabaena sp. pre-tRNA-Leu gene.

In some embodiments, the template DNA comprises a 5′ homology arm and a 3′ homology arm at the 5′ and 3′ ends of the precursor RNA region. The addition of a 5′ homology arm and a 3′ homology arm at the 5′ and 3′ ends of the precursor RNA region may increase the circularization efficiency by bringing the 5′ and 3′ splice sites into proximity of each other, especially when the insert sequence intervening the 3′ Group I intron fragment and the 5′ Group I intron fragment is long. In some embodiments, the 5′ homology arm is from 5 to 50 nucleotides in length, e.g., from 9 to 19 nucleotides in length. In some embodiments, the 3′ homology arm is from 5 to 50 nucleotides in length, e.g., from 9 to 19 nucleotides in length. In some embodiments, the 5′ homology arm and 3′ homology arm are perfectly complement to each other. In other embodiments, the 5′ homology arm and 3′ homology arm are partially (e.g., at least 80%, at least 85%, at least 90%, or at least 95%) complement to each other.

Highly structured sequence, e.g., IRES (internal ribosomal entry site), between the 3′ Group I intron fragment and the 5′ Group I intron fragment may interfere with the folding of the splicing ribozyme, either proximally at the 3′ splice site or distally at the 5′ splice site through long-distance contacts (Wesselhoeft et al., 2018). The addition of a 5′ spacer sequence between the 3′ Group I intron fragment and the insert sequence and/or a 3′ spacer sequence between the insert sequence and the 5′ Group I intron fragment may increase the circularization efficiency, especially when the insert sequence is highly structured. In some embodiments, the DNA template comprises a 5′ spacer sequence between the 3′ Group I intron fragment and the insert sequence. In some embodiments, the 5′ spacer sequence is from 5 to 50 nucleotides in length. In some embodiments, the 5′ spacer sequence is from 10 to 20 nucleotides in length. In certain embodiments, the 5′ spacer sequence is a polyA sequence. In other embodiments, the 5′ spacer sequence is a polyA-C sequence. In some embodiments, the DNA template comprises a 3′ spacer sequence between the insert sequence and the 5′ Group I intron fragment. In some embodiments, the 3′ spacer sequence is from 5 to 50 nucleotides in length. In some embodiments, the 3′ spacer sequence is from 10 to 20 nucleotides in length. In certain embodiments, the 3′ spacer sequence is a polyA sequence. In other embodiments, the 3′ spacer sequence is a polyA-C sequence.

The insert sequence comprises a sequence of interest. The sequence of interest may be a protein coding or noncoding sequence. In some embodiments, the insert sequence comprises a noncoding sequence having a biological activity. Examples of noncoding sequence having a biological activity include, but are not limited to, micro RNA and lnc (long noncoding) RNA.

In some embodiments, the insert sequence comprises a protein coding sequence. The protein coding sequence may encode any protein for therapeutic or diagnostic use. In some embodiments, the protein coding sequence encodes an antibody.

When the insert sequence comprises a protein coding sequence, the insert sequence may further comprise sequences necessary for translation, e.g., an internal ribosomal entry site (IRES) sequence upstream of the protein coding sequence. In some embodiments, the insert sequence comprises an IRES sequence operably connected to a protein coding sequence. As used herein, the phrase “operably connected” means that the IRES sequence is positioned upstream of the protein coding sequence such that the protein coding sequence can be translated into a protein in vivo (inside eukaryotic cells, e.g., human cells) and/or in vitro. The IRES sequence may be any IRES sequence known in the art. In some embodiments, the IRES sequence is selected from an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2, human rhinovirus B, Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Human enterovirus B, Equine rhinitis virus, Ectropis obliqua picoma-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human ATIR, Human BAG-1, Human BCL2, Human BiP, Human c-IAPI, Human c-myc, Human eIF4G, Mouse NDST4L, Human LEF1, Mouse HIF1 alpha, Human n-myc, Mouse Gtx, Human p27kipl, Human PDGF2/c-sis, Human p53, Human Pim-1, Mouse Rbm3, Drosophila reaper, Canine Scamper, Drosophila Ubx, Salivirus, Cosavirus, Parechovirus, Human UNR, Mouse UtrA, Human VEGF-A, Human XIAP, Drosophila hairless, S. cerevisiae TFIID, S. cerevisiae YAP1, Human c-src, Human FGF-1, Simian picomavirus, Turnip crinkle virus, an aptamer to eIF4G, Coxsackievirus B3 (CVB3) or Coxsackievirus A (CVB1/2). In certain embodiments, the IRES sequence is a IRES sequence of Coxsackievirus B3 (CVB3).

In the method of the present invention, the in vitro transcription of the template DNA and the circularization (i.e., self-splicing) of the precursor RNA are carried out in one step. The method does not comprise a step of purifying the precursor RNA before allowing the precursor RNA to self-splice. In other words, the in vitro transcription and the circularization occur in the same reaction solution at the same reaction conditions (e.g., temperature). Therefore, the reaction solution and reaction conditions must be optimized for the efficiency of both in vitro transcription and circularization.

In some embodiments, the reaction solution comprises Mg²⁺ at the concentration greater than 26 mM, e.g., greater than 30 mM or greater than 35 mM. In some embodiments, the concentration of Mg²⁺ in the solution is from 30 mM to 100 mM, e.g., from 30 mM to 90 mM, from 30 mM to 80 mM, from 30 mM to 70 mM, from 30 mM to 60 mM, from 30 mM to 50 mM, from 30 mM to 40 mM, from 35 mM to 100 mM, from 35 mM to 90 mM, from 35 mM to 80 mM, from 35 mM to 70 mM, from 35 mM to 60 mM, from 35 mM to 50 mM, from 35 mM to 40 mM, from 38 to 66 mM, e.g., about 38 mM. In certain embodiments, the concentration of Mg²⁺ in the solution is from 38 mM to 66 mM

In some embodiments, the reaction solution comprises a pyrophosphatase at the concentration of from 1 U/ml to 5 U/ml, e.g., from 1 U/ml to 4 U/ml, from 1.5 U/ml to 3 U/ml, from 1.5 U/ml to 2.5 U/ml, about 1 U/ml, about 2 U/ml, or about 4 U/ml. As used herein, 1 U (unit) of pyrophosphatase is defined as the amount of enzyme that generates 1 μmol of phosphate per minute from inorganic pyrophosphate under standard reaction conditions (a 10 minute reaction at 25° C. in 20 mM Tris-HCl, pH 8.0, 2 mM MgCl₂ and 2 mM PPi).

The reaction solution further comprises ingredients required for in vitro transcription. In some embodiments, the reaction solution comprises an RNA polymerase, an RNase inhibitor, ATP, GTP, CTP, UTP, DTT, and a monovalent cation (Na⁺ or K⁺). In certain embodiments, the reaction solution comprises about 5 U/μl RNA polymerase, about 1 U/μl RNase inhibitor, about 10 mM ATP, about 10 mM GTP, about 10 mM CTP, about 10 mM UTP, about 10 mM DTT, and 5 mM monovalent cation (Na⁺ or K⁺). The reaction solution may comprise a buffer. The pH of the reaction solution may be from 6 to 8, e.g., from 7 to 8, or about 7.5.

The precursor RNA may be unmodified, partially modified or completely modified. In some embodiments, the precursor RNA is unmodified, i.e., contains only naturally occurring nucleotides. In other embodiments, the precursor RNA is partially modified or completely modified. A part or all of at least one ribonucleoside triphosphate in the reaction solution may be replaced with a modified nucleoside triphosphate in order to synthesize partially modified or completely modified precursor RNA. Examples of modified nucleoside triphosphate include, but are not limited to, pseudouridine-5′-triphosphate, 1-methylpseudouridine-5′-triphosphate, 2-thiouridine-5′-triphosphate, 4-thiouridine-5′-triphosphate and 5-methylcytidine-5′-triphosphate.

RNA polymerase used for in vitro transcription may be chosen based on the RNA polymerase promoter in the DNA template. For example, if the RNA polymerase promoter in the DNA template is a T7 virus RNA polymerase promoter, the reaction solution may comprise a T7 RNA polymerase. In some embodiments, the reaction solution comprises an RNA polymerase selected from T7 virus RNA polymerase, T6 virus RNA polymerase, SP6 virus RNA polymerase, T3 virus RNA polymerase, or T4 virus RNA polymerase. In certain embodiments, the RNA polymerase promoter in the DNA template is a T7 virus RNA polymerase and the reaction solution comprises a T7 virus RNA polymerase.

In some embodiments, the in vitro transcription of the template DNA and the circularization (i.e., self-splicing) of the precursor RNA are carried out at a temperature of from 37° C. to 55° C., e.g., from 39° C. to 55° C., from 41° C. to 55° C., from 43° C. to 55° C., from 37° C. to 50° C., from 39° C. to 50° C., from 41° C. to 50° C., from 43° C. to 50° C., from 37° C. to 47° C., from 39° C. to 47° C., from 41° C. to 47° C., from 43° C. to 47° C., from 47° C. to 55° C., from 50° C. to 55° C., from 39° C. to 43° C., about 37° C., about 39° C., about 41° C., about 43° C., about 47° C., about 53° C., or about 55° C. It has been found that the production of a major by-product, dsDNA, is reduced with increasing temperature. dsRNA can be recognized by cytosolic sensors such as RIG-I and MDA5 and then activate the innate immune system (Wu et al., 2020, “Synthesis of low immunogenicity RNA with high-temperature in vitro transcription, RNA 26, 345-360; Olejniczak, 2010, “Sequence-non-specific effects of RNA interference triggers and microRNA regulators, Nucleic Acids Res 38, 1-16). Since ds RNA production should be reduced as much as possible, a temperature higher than 37° C. is preferred. In some embodiments, the in vitro transcription of the template DNA and the circularization (i.e., self-splicing) of the precursor RNA are carried out at a temperature higher than 37° C., e.g., from 39° C. to 55° C., from 41° C. to 55° C., from 43° C. to 55° C., from 39° C. to 50° C., from 41° C. to 50° C., from 43° C. to 50° C., from 39° C. to 47° C., from 41° C. to 47° C., from 43° C. to 47° C., from 47° C. to 55° C., from 50° C. to 55° C., from 39° C. to 43° C., about 39° C., about 41° C., about 43° C., about 47° C., about 53° C., or about 55 CC.

A genetically modified RNA polymerase exhibiting increased thermo stability (e.g., T7 Toyobo) may be preferred if the in vitro transcription of the template DNA and the circularization (i.e., self-splicing) of the precursor RNA are carried out at a high temperature. In some embodiments, the in vitro transcription of the template DNA and the circularization (i.e., self-splicing) of the precursor RNA are carried out at a temperature of from 47° C. to 55° C., e.g., from 50° C. to 55° C., about 47° C., about 53° C., or about 55° C. and the RNA polymerase is a thermostable polymerase (e.g., T7 Toyobo).

The in vitro transcription of the template DNA and the circularization (i.e., self-splicing) of the precursor RNA may be carried out for at least 1 hour, e.g., at least 1.5 hours, at least 2.5 hours, at least 3 hours, from 1 hour to 3 hours, from 1.5 hours to 3 hours, from 2 hours to 3 hours, or from 2.5 hours to 3 hours. The reaction time no less than 1.5 hours is preferred to guarantee the sufficient circularization. On the other hand, the prolongation of the reaction time has the potential to increase by-products. Therefore, the optimal reaction duration of the one-step process may be 2.5-3 hours. In a preferred embodiment, the in vitro transcription of the template DNA and the circularization (i.e., self-splicing) of the precursor RNA are carried out for 2.5-3 hours.

In some embodiments, the method further comprises a step of removing the DNA template after the self-splicing of the RNA. The DNA template may be removed by adding a DNase I, e.g., for 30 min at 37° C.

In some embodiments, the method further comprises a step of purifying the circular RNA after the self-splicing of the RNA or after the step of removing the DNA template, if the method comprises a step of removing the DNA template. In some embodiments, the purification step is selected from a precipitation step, a tangential flow filtration step and a chromatographic step, and a combination thereof. The precipitation step may be an alcoholic precipitation step or LiCl precipitation. The tangential flow filtration step may be a diafiltration step using tangential flow filtration and/or a concentration step using tangential flow filtration. The chromatographic step may be selected from HPLC, anion exchange chromatography, affinity chromatography, hydroxyapatite chromatography, magnetic bead chromatography and core bead chromatography. In some embodiments, the purification step comprises a precipitation step, e.g., LiCl precipitation. In other embodiments, the purification step comprises a chromatography, e.g., magnetic bead chromatography.

### EXAMPLES

### Plasmid Construction

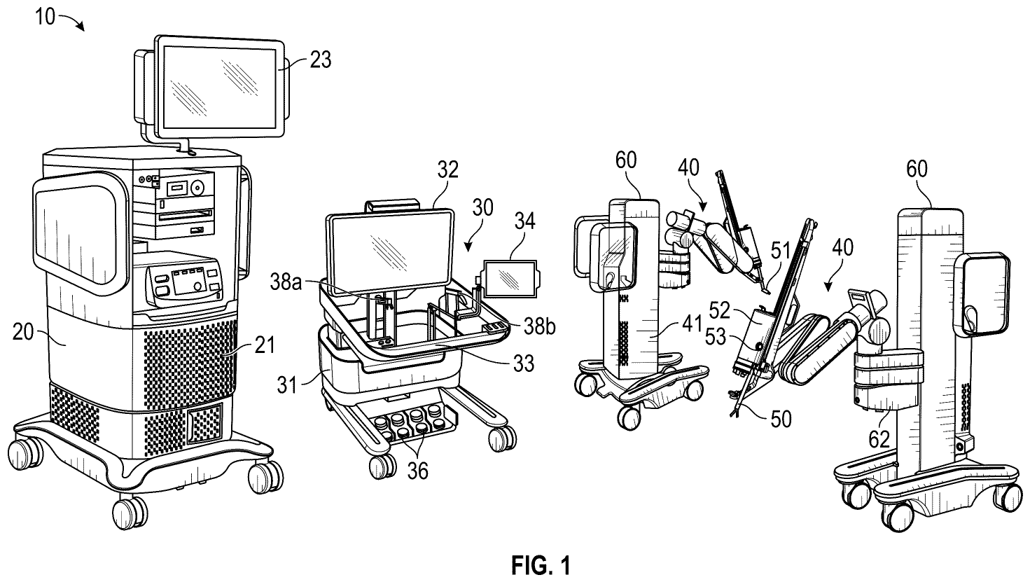

A circRNA precursor sequence-containing plasmid is used as a template for IVT (in vitro transcription). The circRNA precursor sequence is designed based on the group I intron system described in Wesselhoeft et al. (Wesselhoeft et al., 2018, “Engineering circular RNA for potent and stable translation in eukaryotic cells, Nature Communications 9, 2629). An insert sequence containing a Coxsackievirus B3 (CVB3) IRES (internal ribosomal entry site), a GFP sequence and two short regions corresponding to exon fragments (E1 and E2) is flanked by the 3′ and 5′ introns of the permuted group I catalytic intron from Anabaena pre-tRNA gene. The circRNA precursor sequence (SEQ ID NO: 1) is chemically synthesized and cloned into an expression vector containing a T7 polymerase promoter (Genscript). The schematic diagram of the circRNA precursor used in the experiments is shown in FIG. 1.

### Circular RNA Synthesis by the Conventional Procedure

The linearized plasmid DNA is used as a template for in-vitro transcription. The circRNA precursor sequence-containing plasmid is linearized by XbaI enzymatic digestion and circRNA precursors are synthesized by in-vitro transcription from the linearized plasmid DNA template using a T7 RNA polymerase. The reaction mixture (20 μL in total) is prepared as follows: 1 U/μL RNase Inhibitor (Novoprotein E125), 6.67 mM ATP, 20 mM GTP, 6.67 mM CTP, 6.67 mM UTP, 1× Transcription buffer (Novoprotein GMP-EB121 containing 6 mM MgCl₂), 10 mM DTT (Sigma 43816), 4 U/mL Pyrophosphatase Inorganic (Novoprotein GMP-M036), 5 mM NaCl (Invitrogen AM9760G), 20 mM MgCl₂ (Invitrogen M1028), 5 U/L T7 RNA polymerase (Novoprotein GMP-E121), 25 ng/μL linearized plasmid. In-vitro transcription is carried out at 37° C. for 3 hours and then the reaction mixture is treated by DNase I (Novoprotein GMP-E127) for 30 min at 37° C. to remove DNA templates. After DNase I treatment, the synthesized precursor RNA is purified by precipitation with 7.5 M LiCl.

Circularization of the precursor RNA is performed as described in Wesselhoeft et al. with modifications. Circularization reaction (20 μL) is carried out by subjecting 6 μg precursor RNA directly to the circularization reaction. Alternatively, the precursor mRNA is preheated to 70° C. for 5 minutes and immediately placed on ice for 5 minutes. GTP is added to the precursor RNA to a final concentration of 2 mM along with a buffer containing magnesium (50 mM Tris HCl, 10 mM MgCl₂, 1 mM DTT, pH 7.5). The reaction mixture is incubated at 55° C. for 12 minutes, and then the RNA is purified by a column. The RNA products are evaluated by a fragment analyzer.

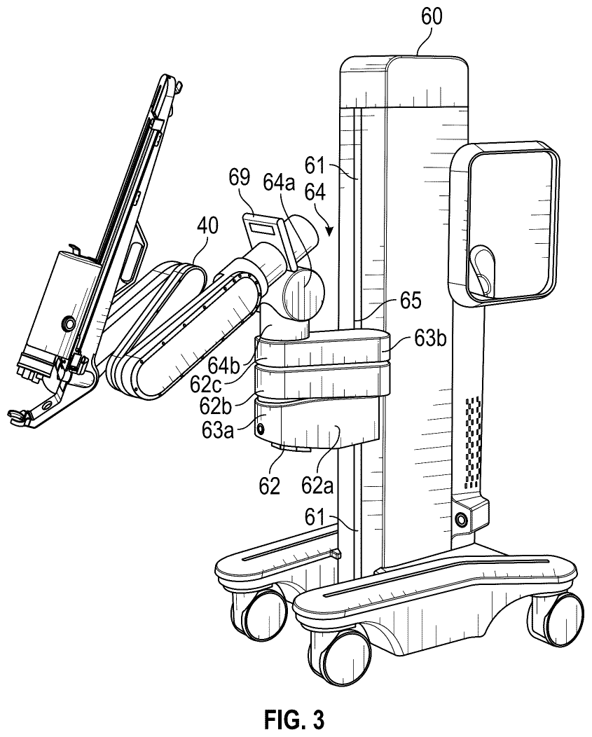

The precursor RNA is circularized through a self-splicing reaction requiring GTP and magnesium ions. This process depends on the structure of group I intron. As shown in FIG. 3, about 60% RNA is circularized when the precursor RNA is subjected directly to the circularization reaction without preheating, while about 70% circularization efficiency is achieved when the precursor RNA is preheated at 70° C., followed by circularization, suggesting that the pre-denaturation of the precursor RNA favors the formation of structures required for the self-splicing of group I intron. These results show that the conventional procedure requires a high-temperature pre-denaturation treatment of LiCl purified IVT products prior to circularization in order to promote the circularization of precursor RNAs.

### Circular RNA Synthesis by the One-Step Process

The linearized plasmid DNA is used as a template for in-vitro transcription. The circRNA precursor sequence-containing plasmid is linearized by XbaI enzymatic digestion. A one-step reaction mixture (20 μL in total) is prepared as follows: 1 U/μL RNase Inhibitor (Novoprotein E125), 10 mM ATP, 10 mM GTP, 10 mM CTP, 10 mM UTP, 1× Transcription buffer (Novoprotein GMP-EB121; containing 6 mM MgCl₂), 10 mM DTT (Sigma 43816), 4 U/mL Inorganic Pyrophosphatases (Novoprotein GMP-M036), 5 mM monovalent cations (Na⁺ or K⁺), MgCl₂ (Invitrogen M1028) ranging from 20 mM to 80 mM, 5 U/μL T7 RNA polymerase (KactusBio GMP-T7P-EE101-12), 25 ng/μL linearized plasmid. The reaction is carried out at 37° C. for 3 hours and then the reaction mixture is treated by DNase I (Novoprotein GMP-E127) for 30 min at 37° C. to remove DNA templates. After DNase I treatment, RNAs are purified by precipitation with 7.5 M LiCl. The RNA products are evaluated by a fragment analyzer.

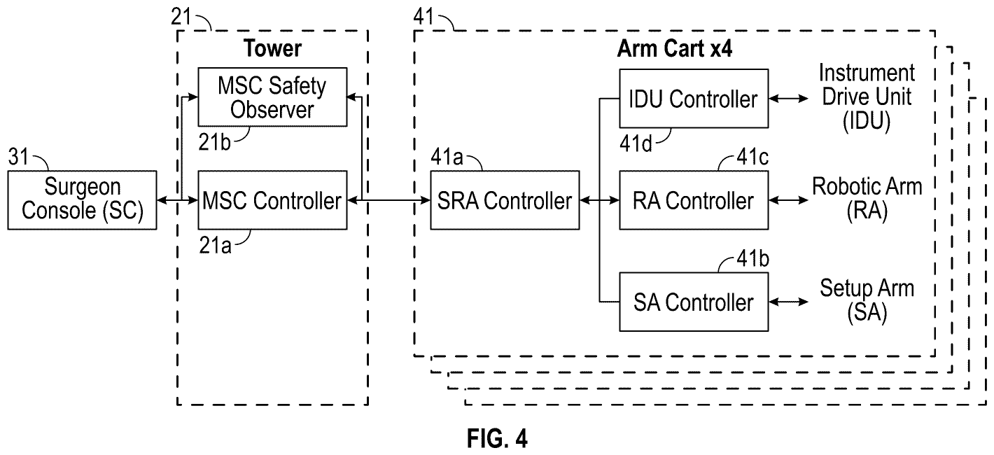

According to the previous study ((Wesselhoeft et al., 2018), the circularization of IVT-derived precursor RNAs through the self-splicing process of group I intron requires 2 mM GTP and 10 mM MgCl₂, while the conventional IVT system contains GTP (10 mM here) and MgCl₂ (26 mM here). FIGS. 4A and 4B show that the synthesized precursor RNAs are not circularized efficiently in the conventional IVT condition (10 mM GTP and 26 mM MgCl₂). Only 7.5% precursor RNAs are circularized in this condition, suggesting that the conventional IVT condition needs to be optimized for the circularization of precursor RNAs.

In order to investigate the effect of Mg²⁺ concentration on the circularization of precursor RNAs, Mg²⁺ concentration is varied, with other components fixed, in the one-step IVT/circularization system (Table 1).

TABLE 1
One-step IVT/circularization system with varied
Mg²⁺ concentration (20 μL)

| Content | Concentration | |
|---|---|---|
| RNase Inhibitor | 1 | U/μL |
| ATP | 10 | mM |
| GTP | 10 | mM |
| CTP | 10 | mM |
| UTP | 10 | mM |
| 1X Transcription buffer | containing 6 mM MgCl₂ | |
| DTT | 10 | mM |
| Pyrophosphatase Inorganic | 4 | U/mL |
| NaCl | 5 | mM |
| MgCl₂ | Variable | |
| T7 RNA polymerase (KactusBio) | 5 | U/μL |
| Linearized plasmid | 25 | ng/μL |

| | |
|---|---|
| Nuclease free water | To 20 μL |

The final Mg²⁺ concentrations tested include 26 mM, 36 mM, 46 mM, 56 mM, 66 mM, and 86 mM, while each NTP concentration is adjusted to 10 mM (A:G:C:U=1:1:1:1). The RNA products are evaluated by a fragment analyzer. The results are shown in FIGS. 4A and 4B. The results show that the increase of Mg²⁺ concentration from 26 mM to 36 mM promotes the circularization of the circRNA precursor. Only 7.5% precursor RNAs are circularized at 26 mM Mg²⁺, while 41.8% precursor RNAs are circularized at 36 mM Mg²⁺. However, further increase of Mg²⁺ concentration up to 86 mM does not change the efficiency of circularization significantly.

The yield of total RNA is also measured in the one-step IVT/circularization system with varied Mg²⁺ concentration (FIG. 4C). Increasing Mg²⁺ concentration from 26 mM to 36 mM not only promotes the circularization of the circRNA precursor but also increase the yield of RNA. However, increasing Mg²⁺ concentration to 86 mM significantly diminishes RNA production.

In the one-step IVT/circularization process, the circularization of precursor RNAs may occur co-transcriptionally and/or post-transcriptionally. The efficiency and yield of circularization at different reaction time points are investigated to address this issue. Except that the Mg²⁺ concentration is adjusted to 38 mM, the other IVT conditions are the same as those in Table 1. The reaction is carried out at 37° C. for 1 h, 1.5 h, 2 h, 2.5 h, 3 h, or 3.5 h, and then the reaction mixture is treated by DNase I (Novoprotein GMP-E127) for 30 min at 37° C. to remove DNA templates. After DNase I treatment, RNAs are purified by precipitation with 7.5 M LiCl.

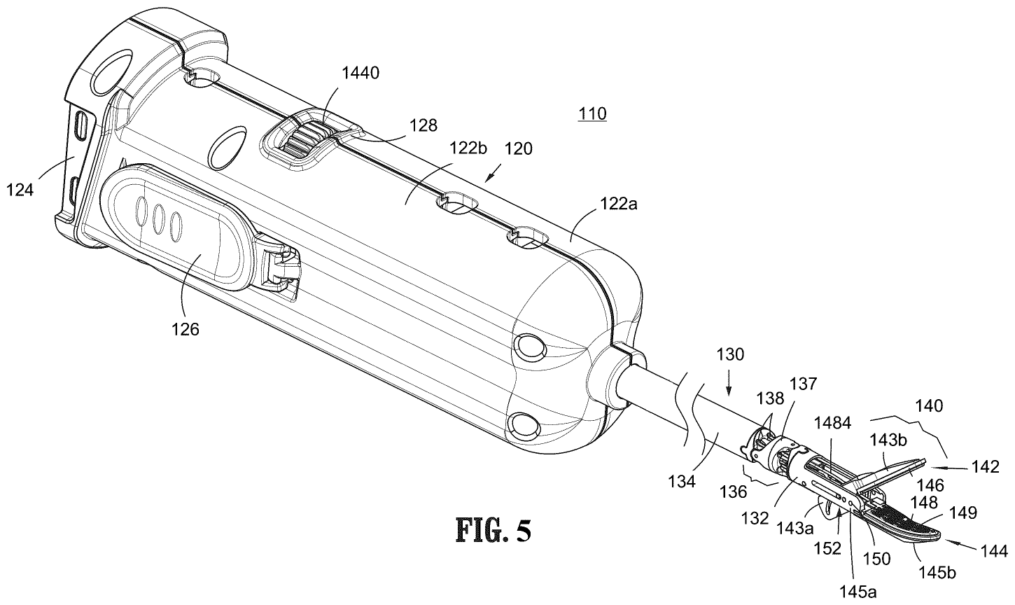

The fragment analysis results show that the extension of the reaction duration up to 2.5 h can improve the efficiency of the circularization of precursor RNAs (FIG. 5A). However, the process is almost completed at 2.5 h. The reaction duration longer than 2.5 h does not increase the circularization efficiency. The effect of the reaction duration on the yield of total RNA is also examined. The results show that the yield exceeds 200 μg/reaction when the reaction is carried out for 1 hr and further extension of the reaction duration does not improve the yield significantly (FIG. 5B). Prolongation of the reaction time has the potential to increase by-products. Therefore, the optimal reaction duration of the one-step process is 2.5-3 hours.

### Optimization of the One-Step Circularization System

To comprehensively evaluate the factors affecting IVT and circularization during the one-step process, a multilevel cross-over experiment is designed using software for four factors: temperature, Mg²⁺ concentration, pyrophosphatase concentration, and reaction temperature. The circularization efficiency is examined by a fragment analyzer and the yield of total RNA is calculated by determining the concentration of the products. The results are shown in Table 2. All the data are inputted into the software for factorial design analysis.

TABLE 2
Factorial design of experiments (DOE) for the one-step circularization system

| | Reaction | | | Inorganic | | Yield/ |
|---|---|---|---|---|---|---|
| Reaction | temperature | Mg²⁺ | | Pyrophosphatases | | reaction |
| order | (° C.) | (mM) | T7 RNAP | (U/mL) | % CircRNA | (ug) |
| 1 | 37 | 26 | T7 KACTUS | 0 | 2.50 | 70.09 |
| 2 | 37 | 26 | T7 KACTUS | 2 | 10.90 | 203.32 |
| 3 | 37 | 38 | T7 KACTUS | 0 | 3.10 | 91.71 |
| 4 | 37 | 38 | T7 KACTUS | 2 | 50.90 | 209.97 |
| 5 | 37 | 66 | T7 KACTUS | 0 | 24.60 | 78.48 |
| 6 | 37 | 66 | T7 KACTUS | 2 | 40.00 | 190.83 |
| 7 | 47 | 26 | T7 KACTUS | 0 | 3.90 | 10.25 |
| 8 | 47 | 26 | T7 KACTUS | 2 | 5.20 | 14.41 |
| 9 | 47 | 38 | T7 KACTUS | 0 | 1.50 | 39.80 |
| 10 | 47 | 38 | T7 KACTUS | 2 | 74.90 | 205.47 |
| 11 | 47 | 66 | T7 KACTUS | 0 | 68.00 | 57.17 |
| 12 | 47 | 66 | T7 KACTUS | 2 | 71.60 | 172.28 |
| 13 | 47 | 26 | T7 KACTUS | 4 | 4.30 | 4.37 |
| 14 | 47 | 38 | T7 KACTUS | 4 | 72.20 | 206.48 |
| 15 | 53 | 26 | T7 KACTUS | 0 | 9.90 | 4.06 |
| 16 | 53 | 26 | T7 KACTUS | 2 | 8.60 | 4.20 |
| 17 | 53 | 38 | T7 KACTUS | 0 | 11.70 | 12.97 |
| 18 | 53 | 38 | T7 KACTUS | 2 | 14.70 | 14.24 |
| 19 | 53 | 66 | T7 KACTUS | 0 | 56.50 | 2.13 |
| 20 | 53 | 66 | T7 KACTUS | 2 | 66.80 | 2.12 |
| 21 | 53 | 26 | T7 KACTUS | 4 | 10.00 | 0.80 |
| 22 | 53 | 38 | T7 KACTUS | 4 | 12.10 | 2.77 |
| 23 | 55 | 26 | T7 KACTUS | 0 | 11.10 | 4.42 |
| 24 | 55 | 26 | T7 KACTUS | 2 | 13.80 | 4.23 |
| 25 | 55 | 38 | T7 KACTUS | 0 | 13.90 | 13.78 |
| 26 | 55 | 38 | T7 KACTUS | 2 | 17.20 | 15.62 |
| 27 | 55 | 66 | T7 KACTUS | 0 | 57.20 | 1.78 |
| 28 | 55 | 66 | T7 KACTUS | 2 | 53.90 | 1.75 |
| 29 | 37 | 26 | T7 TOYOBO | 0 | 2.60 | 7.88 |
| 30 | 37 | 26 | T7 TOYOBO | 2 | 2.20 | 10.45 |
| 31 | 37 | 26 | T7 TOYOBO | 4 | 3.80 | 11.68 |
| 32 | 37 | 38 | T7 TOYOBO | 0 | 3.90 | 53.27 |
| 33 | 37 | 38 | T7 TOYOBO | 2 | 5.40 | 76.11 |
| 34 | 37 | 38 | T7 TOYOBO | 4 | 23.30 | 82.35 |
| 35 | 37 | 66 | T7 TOYOBO | 0 | 42.30 | 18.42 |
| 36 | 37 | 66 | T7 TOYOBO | 2 | 42.70 | 17.56 |
| 37 | 37 | 66 | T7 TOYOBO | 4 | 46.80 | 18.46 |
| 38 | 37 | 76 | T7 TOYOBO | 0 | 14.40 | 9.20 |
| 39 | 37 | 76 | T7 TOYOBO | 2 | 47.40 | 7.73 |
| 40 | 37 | 76 | T7 TOYOBO | 4 | 50.60 | 8.42 |
| 41 | 37 | 96 | T7 TOYOBO | 0 | 50.80 | 2.02 |
| 42 | 37 | 96 | T7 TOYOBO | 2 | 51.80 | 1.66 |
| 43 | 37 | 96 | T7 TOYOBO | 4 | 54.60 | 1.74 |
| 44 | 47 | 26 | T7 TOYOBO | 0 | 3.80 | 6.04 |
| 45 | 47 | 26 | T7 TOYOBO | 2 | 3.40 | 9.02 |
| 46 | 47 | 26 | T7 TOYOBO | 4 | 5.10 | 9.39 |
| 47 | 47 | 38 | T7 TOYOBO | 0 | 5.20 | 69.04 |
| 48 | 47 | 38 | T7 TOYOBO | 2 | 50.70 | 183.11 |
| 49 | 47 | 38 | T7 TOYOBO | 4 | 69.90 | 182.15 |
| 50 | 47 | 66 | T7 TOYOBO | 0 | 60.50 | 84.38 |
| 51 | 47 | 66 | T7 TOYOBO | 2 | 70.50 | 81.95 |
| 52 | 47 | 66 | T7 TOYOBO | 4 | 69.50 | 77.96 |
| 53 | 47 | 76 | T7 TOYOBO | 0 | 68.00 | 42.47 |
| 54 | 47 | 76 | T7 TOYOBO | 2 | 71.30 | 33.79 |
| 55 | 47 | 76 | T7 TOYOBO | 4 | 72.40 | 36.52 |
| 56 | 47 | 96 | T7 TOYOBO | 0 | 70.40 | 8.69 |
| 57 | 47 | 96 | T7 TOYOBO | 2 | 69.70 | 8.25 |
| 58 | 47 | 96 | T7 TOYOBO | 4 | 66.90 | 8.10 |
| 59 | 53 | 26 | T7 TOYOBO | 0 | 6.80 | 3.57 |
| 60 | 53 | 26 | T7 TOYOBO | 2 | 5.90 | 4.97 |
| 61 | 53 | 26 | T7 TOYOBO | 4 | 5.50 | 4.84 |
| 62 | 53 | 38 | T7 TOYOBO | 0 | 6.60 | 56.53 |
| 63 | 53 | 38 | T7 TOYOBO | 2 | 52.00 | 187.99 |
| 64 | 53 | 38 | T7 TOYOBO | 4 | 59.00 | 190.03 |
| 65 | 53 | 66 | T7 TOYOBO | 0 | 50.90 | 83.27 |
| 66 | 53 | 66 | T7 TOYOBO | 2 | 69.70 | 80.46 |
| 67 | 53 | 66 | T7 TOYOBO | 4 | 71.90 | 77.01 |
| 68 | 53 | 76 | T7 TOYOBO | 0 | 68.10 | 46.29 |
| 69 | 53 | 76 | T7 TOYOBO | 2 | 70.50 | 35.76 |
| 70 | 53 | 76 | T7 TOYOBO | 4 | 73.40 | 37.92 |
| 71 | 53 | 96 | T7 TOYOBO | 0 | 69.30 | 8.50 |
| 72 | 53 | 96 | T7 TOYOBO | 2 | 71.00 | 9.48 |
| 73 | 53 | 96 | T7 TOYOBO | 4 | 69.60 | 9.63 |
| 74 | 55 | 26 | T7 TOYOBO | 0 | 5.20 | 2.42 |
| 75 | 55 | 26 | T7 TOYOBO | 2 | 4.10 | 3.48 |
| 76 | 55 | 26 | T7 TOYOBO | 4 | 5.40 | 3.69 |
| 77 | 55 | 38 | T7 TOYOBO | 0 | 6.40 | 48.84 |
| 78 | 55 | 38 | T7 TOYOBO | 2 | 47.30 | 166.11 |
| 79 | 55 | 38 | T7 TOYOBO | 4 | 56.40 | 175.61 |
| 80 | 55 | 66 | T7 TOYOBO | 0 | 57.50 | 73.53 |
| 81 | 55 | 66 | T7 TOYOBO | 2 | 67.80 | 60.41 |
| 82 | 55 | 66 | T7 TOYOBO | 4 | 68.80 | 55.24 |
| 83 | 55 | 76 | T7 TOYOBO | 0 | 67.60 | 31.27 |
| 84 | 55 | 76 | T7 TOYOBO | 2 | 66.50 | 25.58 |
| 85 | 55 | 76 | T7 TOYOBO | 4 | 62.80 | 25.94 |
| 86 | 55 | 96 | T7 TOYOBO | 0 | 62.80 | 7.06 |
| 87 | 55 | 96 | T7 TOYOBO | 2 | 63.30 | 7.85 |
| 88 | 55 | 96 | T7 TOYOBO | 4 | 63.10 | 7.07 |

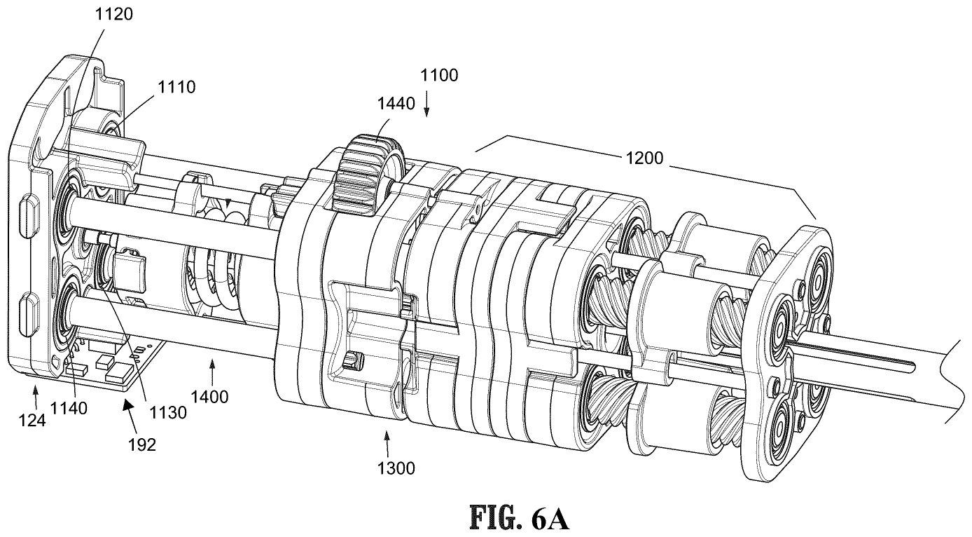

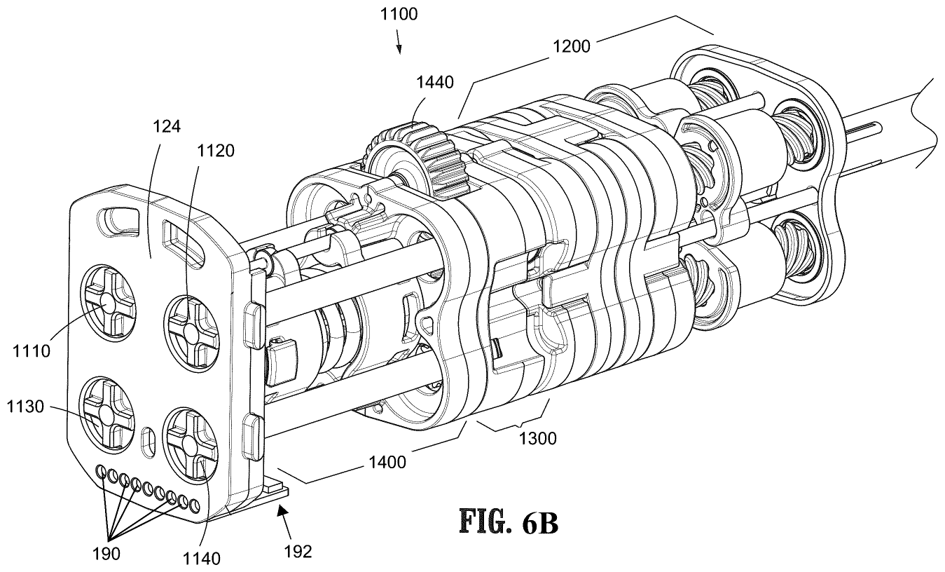

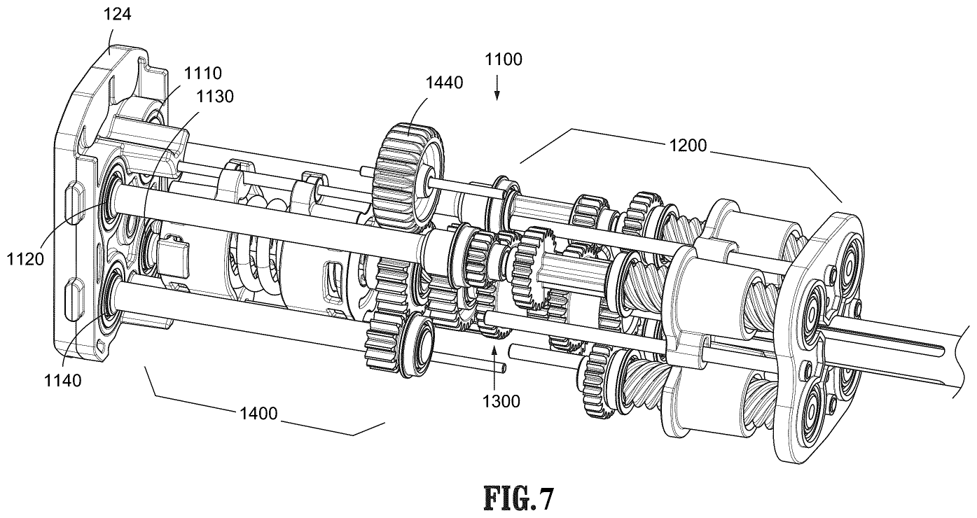

According to the standardized effect analysis, magnesium ion concentration and temperature are the main factors affecting the IVT yield and the interaction between reaction temperature and the type of T7 RNAPs also affects the yield (FIG. 6A). The increase in temperature (greater than 37° C.) decreases the overall IVT yield (FIG. 6B). Increasing Mg²⁺ concentration from 26 mM to 38 mM significantly improved the average IVT yield, but further increase in Mg²⁺ concentration does not increase the average yield under all tested conditions (FIG. 6B). The addition of pyrophosphatase to the reaction increases the yields (FIG. 6B). It is believed that it is because pyrophosphatase helps maintain the magnesium ion concentration stable throughout the IVT process. The type of T7 RNAPs used in IVT has little effect on the average yield of all groups tested, but the effect of temperature on the IVT yield is significantly dependent on which type of T7 RNAPs is used in IVT (FIG. 7). When TOYOBO T7 RNAP (a thermo stable T7 RNA polymerase) is used in IVT, the average IVT yield is the lowest at 37° C. and the average IVT yields at high temperatures (47, 53, or 55° C.) are approximately the same. In contrast, when KACTUS T7 RNAP is used in IVT, the average IVT yield is the highest at 37° C. and the average IVT yields at high temperatures (47, 53, or 55° C.) are reduced greatly, showing that KACTUS T7 RNAP has a reduced enzymatic activity in high temperature conditions.

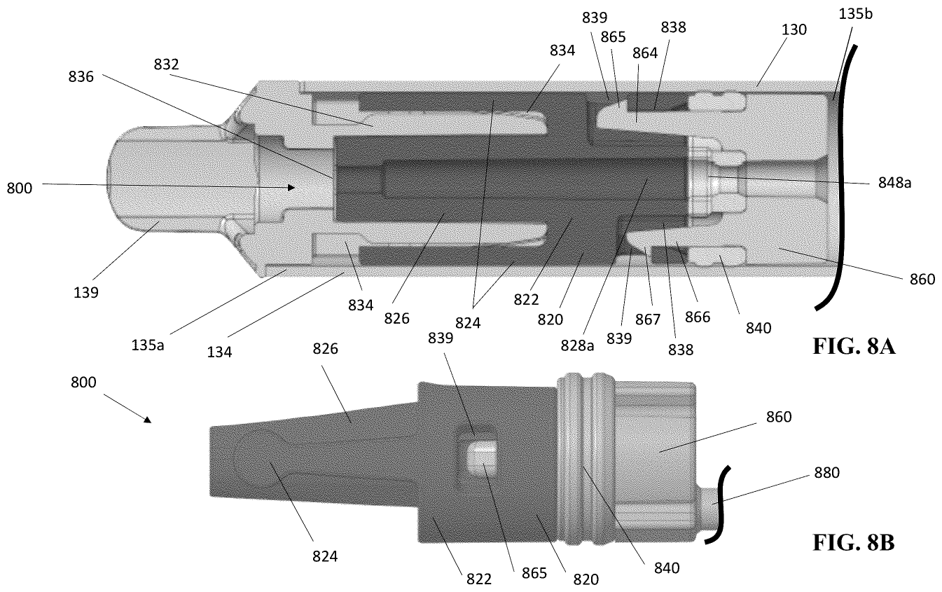

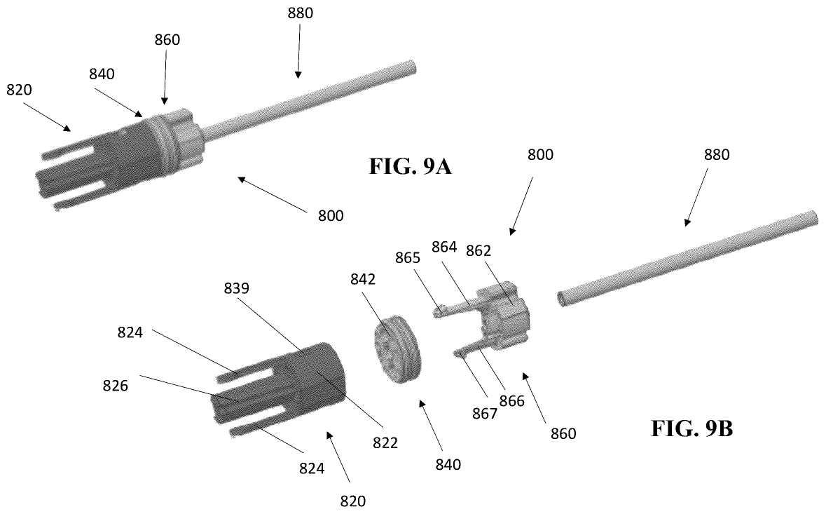

For the circularization efficiency, Mg²⁺ concentration, reaction temperature, pyrophosphatase concentration, and the interaction between Mg²⁺ and pyrophosphatase all affect the circularization of precursor RNAs (FIG. 8A). Increasing the temperature from 37° C. to 47° C. promotes the circularization, but the circularization efficiency is reduced at higher temperatures (53° C. and 55° C.) (FIG. 8B). Increasing Mg²⁺ concentration from 26 Mm to 66 mM promotes the circularization, but higher Mg²⁺ concentrations (76 mM and 96 mM) do not further increase the circularization efficiency (FIG. 8B). The addition of pyrophosphatase to the IVT reaction promotes circularization, but the concentration of pyrophosphatase does not positively correlate with the average circularization efficiency, with the circularization efficiency highest at 2 U/mL pyrophosphatase (FIG. 8B). It is worth noting that the circularization can be further promoted when Mg²⁺ is combined with pyrophosphatase (FIG. 9). After multifactorial optimization, the one-step process can achieve the circularization efficiency similar to the conventional process. For example, when adjusting Mg²⁺ concentration to 38 mM, the concentration of pyrophosphatase to 2 U/mL, and the reaction temperature to 47° C., the circularization efficiency can be as high as 74.9% (Table 2).

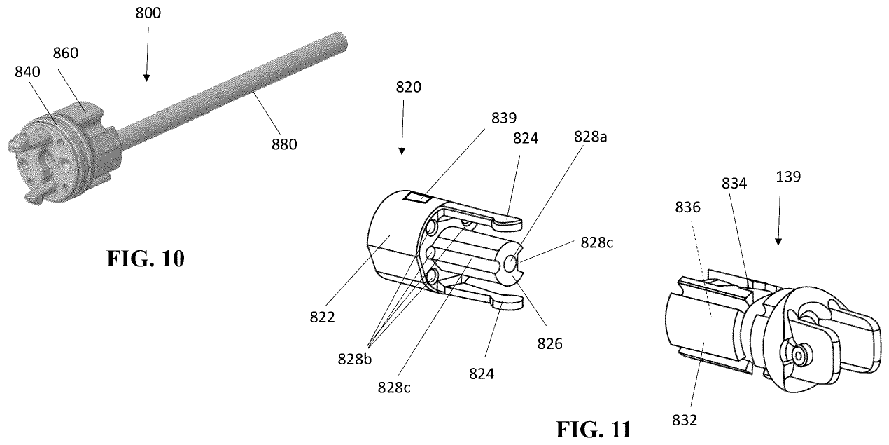

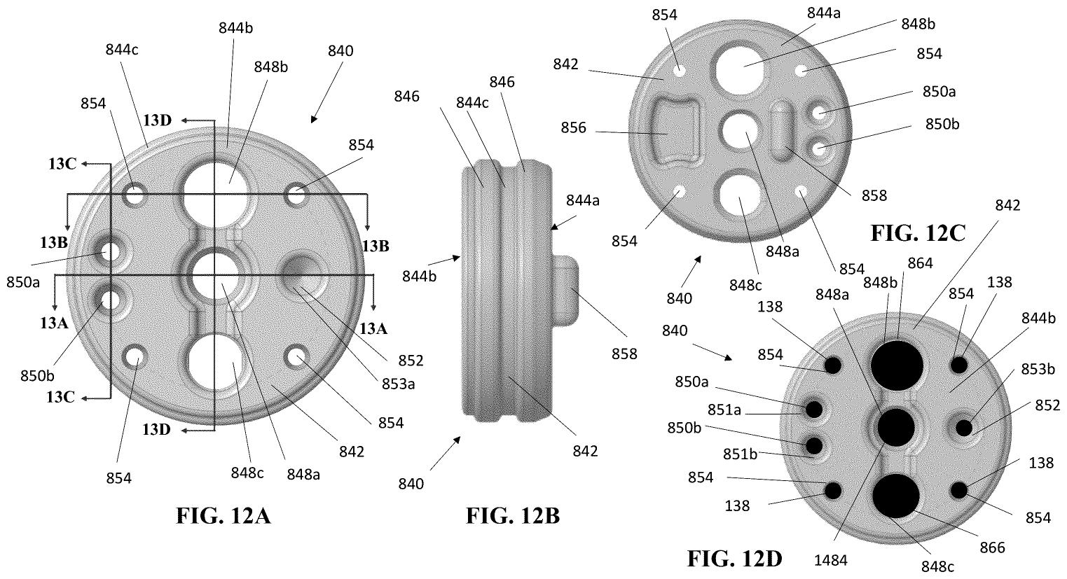

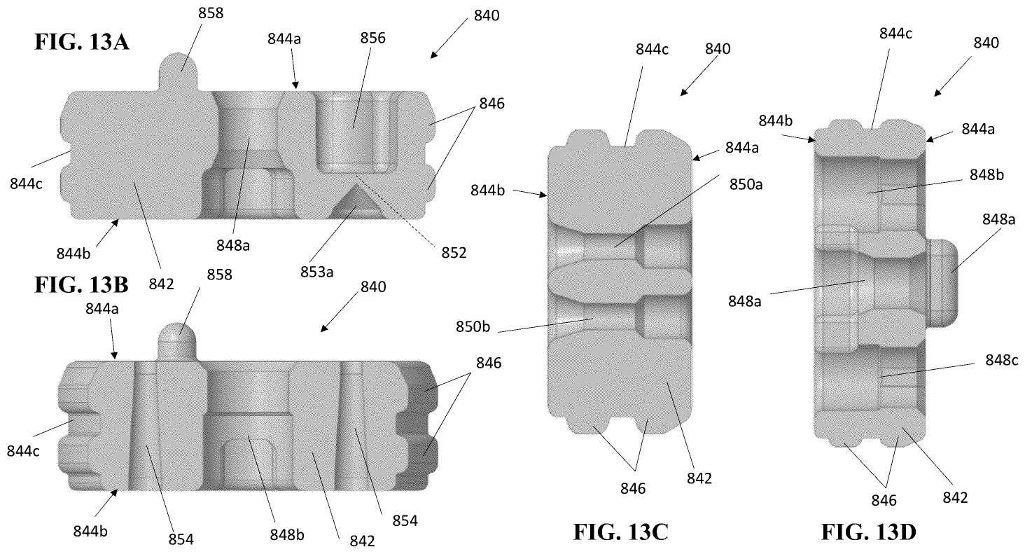

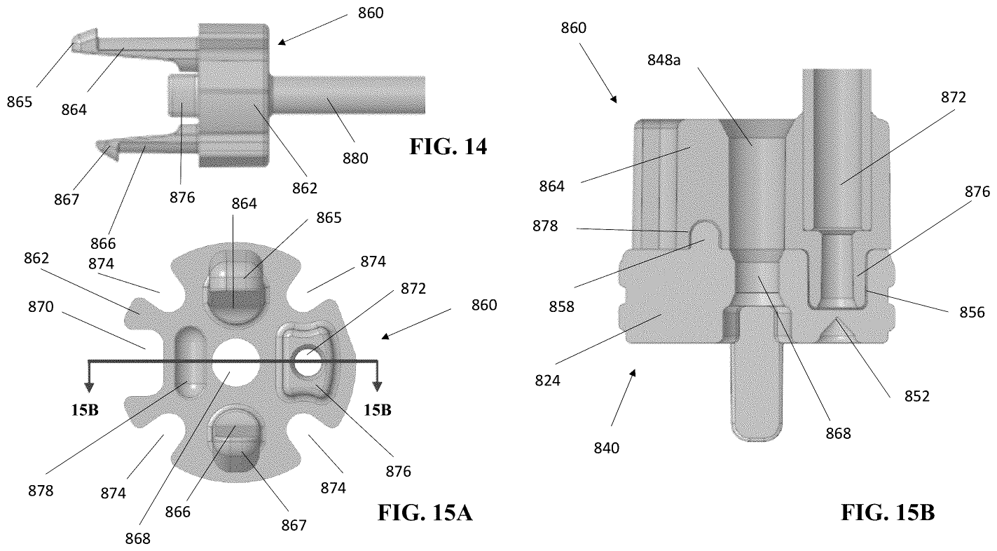

One major by-product identified in the one-step process is dsRNA, which can be recognized by cytosolic sensors, such as RIG-I and MDA5, and then activate the innate immune system. The amount of dsRNAs produced from the IVT conditions of various temperatures and magnesium concentrations are examined. An antibody-dependent Fluorescence Resonance Energy Transfer (FRET) assay is used to detect the dsRNAs in IVT samples. The specific procedures are performed according to the kit manual (Cisbio 64RNAPEG). The results are shown in Table 3 and FIG. 10. The results show that temperature is a factor that directly affects dsRNA production (Table 3 and FIG. 10). The dsRNA production is suppressed with increasing temperature, while there is no significant effect of Mg²⁺ concentration on dsRNA production.

TABLE 3
Effects of temperature and Mg²⁺ concentration on dsRNA content

| Reaction | | |
|---|---|---|
| temperature (° C.) | Mg²⁺(mM) | % dsRNA |
| 37 | 24 | 0.16 |
| 37 | 26 | 0.16 |
| 37 | 28 | 0.16 |
| 37 | 30 | 0.18 |
| 37 | 32 | 0.17 |
| 37 | 34 | 0.16 |
| 37 | 36 | 0.16 |
| 37 | 38 | 0.14 |
| 39 | 24 | 0.12 |
| 39 | 26 | 0.12 |
| 39 | 28 | 0.14 |
| 39 | 30 | 0.14 |
| 39 | 32 | 0.14 |
| 39 | 34 | 0.14 |
| 39 | 36 | 0.12 |
| 39 | 38 | 0.11 |
| 41 | 24 | 0.1 |
| 41 | 26 | 0.11 |
| 41 | 28 | 0.11 |
| 41 | 30 | 0.11 |
| 41 | 32 | 0.1 |
| 41 | 34 | 0.1 |
| 41 | 36 | 0.08 |
| 41 | 38 | 0.09 |
| 43 | 24 | 0.11 |
| 43 | 26 | 0.09 |
| 43 | 28 | 0.08 |
| 43 | 30 | 0.09 |
| 43 | 32 | 0.08 |
| 43 | 34 | 0.08 |
| 43 | 36 | 0.07 |
| 43 | 38 | 0.07 |

While the disclosure has been described with respect to specific examples including presently preferred modes of carrying out the disclosure, those skilled in the art will appreciate that there are numerous variations and permutations of the above described systems and techniques. It is to be understood that other embodiments may be utilized and structural and functional modifications may be made without departing from the scope of the present disclosure. Thus, the scope of the disclosure should be construed broadly as set forth in the appended claims.

### Sequencing List

SEQ ID No: 1: Precursor sequence
TAATACGACTCACTATAGGGAGACCCTCGACCGTCGATTGTCCACTGGT
CAACAATAGATGACTTACAACTAATCGGAAGGTGCAGAGACTCGACGGG
AGCTACCCTAACGTCAAGACGAGGGTAAAGAGAGAGTCCAATTCTCAAA
GCCAATAGGCAGTAGCGAAAGCTGCAAGAGAATGAAAATCCGTTGACCT
TAAACGGTCGTGTGGGTTCAAGTCCCTCCACCCCCACGCCGGAAACGCA
ATAGCCGAAAAACAAAAAACAAAAAAAACAAAAAAAAAACCAAAAAAAC
AAAACACATTAAAACAGCCTGTGGGTTGATCCCACCCACAGGCCCATTG
GGCGCTAGCACTCTGGTATCACGGTACCTTTGTGCGCCTGTTTTATACC
CCCTCCCCCAACTGTAACTTAGAAGTAACACACACCGATCAACAGTCAG
CGTGGCACACCAGCCACGTTTTGATCAAGCACTTCTGTTACCCCGGACT
GAGTATCAATAGACTGCTCACGCGGTTGAAGGAGAAAGCGTTCGTTATC
CGGCCAACTACTTCGAAAAACCTAGTAACACCGTGGAAGTTGCAGAGTG
TTTCGCTCAGCACTACCCCAGTGTAGATCAGGTCGATGAGTCACCGCAT
TCCCCACGGGCGACCGTGGCGGTGGCTGCGTTGGCGGCCTGCCCATGGG
GAAACCCATGGGACGCTCTAATACAGACATGGTGCGAAGAGTCTATTGA
GCTAGTTGGTAGTCCTCCGGCCCCTGAATGCGGCTAATCCTAACTGCGG
AGCACACACCCTCAAGCCAGAGGGCAGTGTGTCGTAACGGGCAACTCTG
CAGCGGAACCGACTACTTTGGGTGTCCGTGTTTCATTTTATTCCTATAC
TGGCTGCTTATGGTGACAATTGAGAGATCGTTACCATATAGCTATTGGA
TTGGCCATCCGGTGACTAATAGAGCTATTATATATCCCTTTGTTGGGTT
TATACCACTTAGCTTGAAAGAGGTTAAAACATTACAATTCATTGTTAAG
TTGAATACAGCAAAATGGTGAGCAAGGGCGAGGAGCTGTTCACCGGGGT
GGTGCCCATCCTGGTCGAGCTGGACGGCGACGTAAACGGCCACAAGTTC
AGCGTGTCTGGCGAGGGCGAGGGCGATGCCACCTACGGCAAGCTGACCC
TGAAGTTCATCTGCACCACCGGCAAGCTGCCCGTGCCCTGGCCCACCCT
CGTGACCACCCTGACCTACGGCGTGCAGTGCTTCAGCCGCTACCCCGAC
CACATGAAGCAGCACGACTTCTTCAAGTCCGCCATGCCCGAAGGCTACG
TCCAGGAGCGCACCATCTTCTTCAAGGACGACGGCAACTACAAGACCCG
CGCCGAGGTGAAGTTCGAGGGCGACACCCTGGTGAACCGCATCGAGCTG
AAGGGCATCGACTTCAAGGAGGACGGCAACATCCTGGGGCACAAGCTGG
AGTACAACTACAACAGCCACAACGTCTATATCATGGCCGACAAGCAGAA
GAACGGCATCAAGGCGAACTTCAAGATCCGCCACAACATCGAGGACGGC
AGCGTGCAGCTCGCCGACCACTACCAGCAGAACACCCCCATCGGCGACG
GCCCCGTGCTGCTGCCCGACAACCACTACCTGAGCACCCAGTCCGCCCT
GAGCAAAGACCCCAACGAGAAGCGCGATCACATGGTCCTGCTGGAGTTC
GTGACCGCCGCCGGGATCACTCTCGGCATGGACGAGCTGTACAAGTGAT
AAAAAAAACAAAAAACAAAACGGCTATTATGCGTTACCGGCGAGACGCT
ACGGACTTAAATAATTGAGCCTTAAAGAAGAAATTCTTTAAGTGGATGC
TCTCAAACTCAGGGAAACCTAAATCTAGTTATAGACAAGGCAATCCTGA
GCCAAGCCGAAGTAGTAATTAGTAAGACCAGTGGACAATCGACGGATAA
CAGCATATCTAGA

1. A method of preparing a circular RNA, comprising providing a template DNA, wherein the template DNA comprises a sequence encoding a precursor RNA, in a reaction solution to permit synthesis of the precursor RNA by in vitro transcription of the template DNA and allowing the precursor RNA to self-splice, thereby producing a circular RNA, wherein the in vitro transcription of the template DNA and the self-splicing of the precursor RNA are carried out in the same reaction solution under the same reaction conditions.

2. The method of claim 1, wherein the method does not comprise a step of purifying the precursor RNA before allowing the precursor RNA to self-splice.

3. The method of claim 1, wherein the DNA template comprises the following elements operably connected to each other and arranged in the following sequence: an RNA polymerase promoter, optionally a 5′ homology arm, a 3′ Group I intron fragment containing a 3′ splice site dinucleotide, optionally a 5′ spacer sequence, an insert sequence, optionally a 3′ spacer sequence, a 5′ Group I intron fragment containing a 5′ splice site dinucleotide, and optionally a 3′ homology arm.

4. The method of claim 3, wherein the insert sequence comprises a protein coding sequence, and wherein the insert sequence comprises an IRES sequence operably connected to the protein-coding sequence.

5. The method of claim 1, wherein the reaction solution comprises Mg²⁺ at the concentration greater than 26 mM.

6. The method of claim 1, wherein the reaction solution comprises Mg²⁺ at the concentration greater than 35 mM, optionally wherein the concentration of Mg²⁺ in the solution is from 38 mM to 66 mM.

7. The method of claim 1, wherein the reaction solution comprises a pyrophosphatase at the concentration of from 1 U/ml to 5 U/ml, from 1 U/ml to 4 U/ml, from 1.5 U/ml to 3 U/ml, from 1.5 U/ml to 2.5 U/ml, about 1 U/ml, about 2 U/ml, or about 4 U/ml.

8. The method of claim 1, wherein the reaction solution comprises 38-66 mM Mg²⁺, optionally 1-4 U/ml pyrophosphatase, an RNA polymerase, an RNase inhibitor, ATP, GTP, CTP, UTP, DTT, and a monovalent cation (Na⁺ or K⁺).

9. The method of claim 1, wherein the in vitro transcription of the template DNA and the circularization (i.e., self-splicing) of the precursor RNA are carried out at a temperature of from 37° C. to 55° C.

10. The method of claim 1, the in vitro transcription of the template DNA and the circularization (i.e., self-splicing) of the precursor RNA are carried out at a temperature higher than 37° C., optionally wherein the in vitro transcription of the template DNA and the circularization (i.e., self-splicing) of the precursor RNA are carried out at a temperature of from 39° C. to 50° C.

11. The method of claim 1, wherein the in vitro transcription of the template DNA and the circularization (i.e., self-splicing) of the precursor RNA are carried out for at least 1 hour, optionally wherein the in vitro transcription of the template DNA and the circularization (i.e., self-splicing) of the precursor RNA are carried out for 2.5-3 hours.

12. The method of claim 1, wherein the method further comprises a step of removing the DNA template after synthesis of the precursor RNA, optionally wherein the DNA template is removed by adding a DNase I, e.g., for 30 min at 37° C.

13. The method of claim 12, wherein the method further comprises a step of purifying the circular RNA after the step of removing the DNA template.

14. The method of claim 13, wherein the purification step is selected from a precipitation step, a tangential flow filtration step and a chromatographic step, and a combination thereof.

15. A reaction solution for use in a one-step circular RNA synthesis, which is an aqueous solution comprising Mg²⁺ in a concentration of greater than 26 mM, optionally a pyrophosphatase (e.g., 1-4 U/ml pyrophosphatase), an RNA polymerase, an RNase inhibitor, nucleoside triphosphates (e.g., ATP, GTP, CTP, and UTP), a reducing agent (e.g., DTT), and a monovalent cation (e.g., selected from Na⁺, K⁺, and combinations thereof).

16. A reaction solution for use in a method according to claim 1, which is an aqueous solution comprising Mg2+ in a concentration of greater than 26 mM, optionally a pyrophosphatase (e.g., 1-4 U/ml pyrophosphatase), an RNA polymerase, an RNase inhibitor, nucleoside triphosphates (e.g., ATP, GTP, CTP, and UTP), a reducing agent (e.g., DTT), and a monovalent cation (e.g., selected from Na+, K+, and combinations thereof).


SEQUENCE LISTING (1 sequence)

SEQ ID NO: 1 - DNA, 1973 bp, synthetic construct
  source 1..1973: mol_type = other DNA
taatacgactcactatagggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctaga